(19) United States
(12) Patent Application Publication
(10) Pub. No.: US 2022/0386573 A1
(43) Pub. Date: Dec. 8, 2022

(54) HUMANIZED MOUSE MODEL WITH HUMAN IMMUNE SYSTEM

(71) Applicant: Board of Regents, The University of Texas System, Austin, TX (US)

(72) Inventors: Daniel Chupp, San Antonio, TX (US); Hong Zan, San Antonio, TX (US); Paolo Casali, San Antonio, TX (US)

(21) Appl. No.: 17/776,856

(22) PCT Filed: Nov. 13, 2020
(86) PCT No.: PCT/US2020/060409

§ 371 Date: May 13, 2022

Related U.S. Application Data
(60) Provisional application No. 62/935,708, filed on Nov. 15, 2019.

(51) Int. Cl. A01K 67/027

(52) U.S. Cl. CPC A01K 67/0271; A01K 2207/12; A01K 2227/105; A01K 2267/03

(57) ABSTRACT

Described herein are transgenic mice for testing immunogenicity and protective efficacy of a wide variety of therapeutic agents and vaccines, determining allograft rejection, and developing monoclonal antibodies and generating hybridomas. Methods of generating a transgenic mouse is also described. Described herein are mouse models capable of expressing B cell, a T cell, a monocyte, a macrophage, a dendritic cell, a NK cell, a iNKT cell, an innate lymphoid cell, a microglia cell, a red blood cell, which can develop into a functional human immune system.

### CROSS REFERENCE TO RELATED APPLICATIONS

This application claims the benefit of priority of the filing date of U.S. Provisional Application No. 62/935,708, filed on Nov. 15, 2019. The content of this earlier filed application is hereby incorporated by reference in its entirety.

### STATEMENT REGARDING FEDERALLY FUNDED RESEARCH

This invention was made with government support under grant nos. AI079705, AI105813, and AI138944 awarded by the National Institutes of Health. The government has certain rights in the invention.

### BACKGROUND

Animal models are tools in biomedical research with mice being one of the most widely used surrogates of human biology. Although mouse models recapitulate many characteristics of human biological systems, certain aspects are inconsistent with human biology, particularly in the immune system. These divergences include differential TLR expression, species-specific pathogenesis, immune responses, and drug interactions. Traditionally, human studies have been limited to ex vivo and in vitro analyses or costly clinical trials. Thus, underscoring the need for an in vivo model that faithfully recapitulates the human immune system.

### BRIEF DESCRIPTION OF THE DRAWINGS

FIGS. 1A-G show NSGW41 H-Mice support greater engraftment and long-term persistence of human immune reconstitution than NSG mice. FIG. 1A shows the levels of human immune reconstitution assessed biweekly by flow cytometry analysis of peripheral blood cells expressing either human or mouse CD45. Engraftment levels depicted as human CD45+ percent of total CD45+ cells. FIG. 1B is a representative FACS plots of both huNSG and huNSG/cKitW41J mice at 10, 20, and 30 weeks of age. FIG. 1C is a representative FACS plot of NSGW41 H-Mice reaching greater than 95% (96.4% of total CD45+ expressing cells) peripheral blood human CD45+ reconstitution. FIG. 1D shows huNSG, huNSG/cKitW41J and NSGW41 H-Mice human hematopoietic lineage cell counts. FIG. 1E shows the proportions of B cells and T cells within the peripheral blood, spleen, and lymph nodes of huNSG, huNSG/cKitW41J and NSGW41 H-Mice assessed by percent of human CD45+ cells. FIG. 1F shows the peripheral blood human leukocyte differentiation kinetics of huNSG, huNSG/KitW41J and NSGW41 H-Mice depicted as cells per ml at 10, 15, and 20 weeks post-engraftment. FIG. 1G shows the proportion of CD34+ cells within the bone marrow 6 and 25 weeks post-engraftment.

FIGS. 2A-D shows the induction of CSR and plasma cell differentiation in response to in vitro stimulation. B cells were isolated from a healthy adult and NSGW41 H-mice by positive selection and were stimulated for 120 h with three different activation conditions. FIG. 2A shows human donor and NSGW41 H-mice B cells stimulated with CD154 plus IL-2, IL-4 and IL-21. FIG. 2B shows the comparison of B cells isolated from healthy adult donor and H-mice stimulated with CpG, IL-2, IL-21, TGF-β, and retinoic acid (RA). FIG. 2C shows human B cells from both healthy adult donors and NSGW41 H-Mice stimulated with CpG, IL-2, IL-4, IL-21. FIG. 2D shows the relative expression of AICDA, PRDMJ, and post-recombination transcripts within human donor B cells and those isolated from NSGW41 H-Mice following stimulation with above conditions.

FIGS. 3A-F shows NSGW41 H-Mice mount a mature antibody response to both T-dependent and T-independent antigens. Jax CD34⁺, HuNSG/Kit^{W41J}, and NSGW41 H-mice were injected intraperitoneally with NP₁₆-CGG (100 μg) in PBS (100 μl) with alum. FIG. 3A shows total IgM, IgG, IgA, and IgE as well as NP-binding IgM, IgG and IgA titers analyzed 28 days following immunization by ELISA and depicted as relative units (RU). FIG. 3B shows the proportion of class-switched IgG and IgA B cells as well as CD27⁺ CD38⁺ plasma cells and IgD CD27⁺ memory B cells in spleen and lymph nodes analyzed by flow cytometry 28 days following NP₁₆-CGG (100 μg) injection. FIG. 3C shows Jax CD34⁺, HuNSG/Kit^{W41J}, and NSGW41 H-mice injected intraperitoneally with DNP-CpG (25 μg) in PBS (100 μl). Total IgM, IgG, IgA, and IgE titers and DNP-binding IgM, IgG, and IgA titers in serum analyzed by ELISA at day 28 and depicted as relative (RU). FIG. 3D shows the proportion of class-switched IgG and IgA B cells as well as CD27⁺ CD38⁺ plasma cells, and Ig D CD27⁺ memory B cells in spleen and lymph nodes were analyzed by flow cytometry 28 days following DNP-CpG injection.

FIGS. 4A-D show E2 pre-conditioning promotes secondary lymphoid development and reconstitution, and increased myeloid lineage differentiation. FIG. 4A shows spleen and lymph nodes from unengrafted NSG and engrafted huNSG, Jax CD34⁺, NSGW41, and NSGW41 H-Mice. Following estrogen pre-conditioning, NSGW41 H-Mice exhibit expanded spleen and lymph node development and reconstitution of total mononuclear cells, and increased numbers of human B, T NK cells, dendritic cells, and monocytes/macrophages. FIG. 4B shows the proportion of human CD45⁺ cells in the spleen and lymph nodes. FIG. 4C shows the proportion of TFB cells within the spleen 28 days after immunization. FIG. 4D shows the absolute number of TFH cells within the spleen. FIG. 4E shows the proportion of TFH cells within the lymph nodes 28 days after immunization.

FIGS. 5A-D show E2 pre-conditioning promotes B2 proliferation, memory B cell and germinal center formation. FIG. 5A shows the proportion of human B1 cells within the peripheral blood before (day 0) and peripheral blood and spleen 35 days after immunization with NP16-CGG (100 μg) in PBS (100 μl) with alum in Jax CD34+, HuNSG/KitW41J, and H-mice. FIG. 5B shows the proportion of memory B cells, identified as CD38− IgD− CD27+, within the peripheral blood 90 days following primary injection and germinal center B cells, identified as CD19+ CD20+ CD38+, within the lymph nodes harvested 10 days following primary injection of NP16-CGG (100 μg) in PBS (100 μl) with alum in either untreated (HuNSGW41J mice) or estrogen pre-conditioned (NSGW41 H-Mice). FIG. 5C shows photomicrographs of hematoxylin- and eosin-stained spleens. Images are representative of three individual mice per group. Spleens were harvested 10 days after NP-CGG immunization and prepared for histology. FIG. 5D shows paraffin immunohistochemistry sections of spleen were stained with anti-CD20, anti-CD3, and anti-KI67.

FIGS. 6A-F show the characterization of autoimmune pathophysiology. FIG. 6A shows titers of total human IgM, IgG and IgA as well as IgG autoantibodies analyzed by ELISA. FIG. 6B shows autoantibody production assessed by ANA staining of serum collected from NSGW41 H-Mice (Nil), pristane injected mice without treatment (Hu-lupus Nil), and pristane injected mice treated with epigenetic modulator (Hu-lupus EM). FIG. 6C shows kidney histology sections stained with H&E from NSGW41 H-Mice, Hu-lupus Nil, and Hu-lupus EM mice. FIG. 6D shows anti-IgG immunofluorescent stained kidney sections from NSGW41 H-Mice, Hu-lupus Nil, and Hu-lupus EM mice. FIG. 6E shows H-lupus Mice survival over 6 weeks following induction of autoimmunity. FIG. 6F shows photos of NSGW41 H-Mice and H-Lupus Mice 6 weeks post-pristane.

FIGS. 7A-D shows reduced CSR, AID expression and plasma cell differentiation in Autoimmune induced H-Lupus mice. FIG. 7A shows the proportion of IgM⁺, IgD⁺, IgG⁺ and IgA⁺ B cells within the lymph nodes assessed by flow cytometry in both NSGW41 H-Mice and pristane injected H-lupus mice. FIG. 7B shows AID expression in splenocytes and FIG. 7C shows the proportion of CD27+ CD38+ plasma cells in the spleen and bone marrow from NSGW41 H-Mice and H-Lupus mice analyzed by flow cytometry.

FIGS. 8A-D show NSGW41 H-Mice mount class-switch, high-affinity antibodies to bacterial and viral antigens. Anti-flagellin immunoglobulins in immune sera from NSGW41 H-Mice mice display complement-mediated serum bactericidal antibody activity against S. typhimurium in vitro. NSGW41 H-Mice (18 weeks old) were injected intraperitoneally with S. typhimurium flagellin (50 μg) in alum (100 μl) at day 0 and flagellin (50 μg) in PBS (100 μl) at Day 7. Sera was collected prior to and 21 days after primary injection. FIG. 8A shows sera were two-fold serially diluted for titration of flagellin-binding and total IgM, IgG and IgA, by specific ELISAs using plates coated with purified flagellin (1 μg/ml) or unlabeled IgM, IgG and IgA (1 μg/ml), respectively. Flagellin-binding antibody titers are depicted as relative units (RU). FIG. 8B shows in vitro serum bactericidal activity assay with two-fold serially diluted sera from unimmunized NSGW41 H-Mice, flagellin-immunized NSGW41 H-Mice, and sera positive human donors. FIG. 8C shows IgM and IgG RBD-binding titers 28 days following immunization with recombinant SARS-CoV-2 S1 spike protein RBD domain. FIG. 8D shows binding curves of IgM and IgG RBD-binding titers 28 days following immunization.

FIG. 9 shows CDR3 lengths in recombined Ig VHDJH gene segments of human and NSGW41 H-Mice. B cells. Recombined VHDJH-CH transcripts were amplified using forward (degenerate) primers for leader sequences of (VH1 and VH3 family genes) in conjunction with reverse Cγ isotype-specific primers. The average percentage of total sequences at any given CDR3 amino acid (aa) length in recombined VHDJH transcripts expressed by CD19+IgG+ B cells from 3 healthy human subjects and 3 NSGW41 H-Mice is depicted.

FIGS. 10A-C show somatic point-mutations in recombined Ig VHDJH gene segments of NSGW41 H-Mice. Recombined VHDJH-CH transcripts were amplified using forward (degenerate) primers for leader sequences of (VH1 and VH3 family genes) in conjunction with reverse Cγ isotype-specific primers. Somatic point-mutations in recombined Ig VHDJH transcripts expressed by CD19+IgG+ B cells with (FIG. 10A), pie charts depict the proportions of sequences that carry different numbers of point mutations; listed below the pie charts is the overall mutation frequency (change/base). Donut charts depict the spectrum of point-mutations. FIG. 10B shows scatter plots depicting distribution of mutations (change/base) across each transcript recombined Ig VHDJH transcripts. FIG. 10C shows histograms depicting average mutational frequencies. Frequency of silent and replacement point-mutations in framework regions (FR) and complementarity-determining regions (CDRs) in 3 NSGW41 H-Mice.

FIG. 11 show that NSGW41 H-Mice B cells undergo robust clonal expansion following immunization. Following immunization of NSGW41 H-Mice, recombined VHDJH-CH transcripts were amplified using forward (degenerate) primers for leader sequences of (VH1 and VH3 family genes) in conjunction with reverse Cγ isotype-specific primers. VHDJH-Cγ transcripts were grouped based on identical CDR3 sequence and identical VHDJH gene segment usage. Size of the rectangle in box graph represents the number of unique transcripts from each clone.

FIG. 12 shows that NSGW41 H-Mice B cells undergo complex clonal expansion. Recombined VHDJH-CH transcripts were amplified using forward (degenerate) primers for leader sequences of (VH1 and VH3 family genes) in conjunction with reverse Cγ isotype-specific primers. Phylogenic mapping demonstrates complex clonal expansion from unmutated progenitors.

### SUMMARY

Disclosed herein are genetically modified mice comprising: a) a loss-of-function mutation in the gene that encodes for the protein kinase, DNA-activated, catalytic polypeptide; and b) a loss-of-function mutation in the gene that encodes for the interleukin 2 receptor α; wherein the mouse further comprises an engraftment of human hematopoietic stem cells.

Disclosed herein are methods of making a mouse with a human immune system, the method comprising: engrafting a mouse with human hematopoietic cells, wherein the engrafting is intracardial, wherein the mouse comprises: a) a loss-of-function mutation in the gene that encodes for the protein kinase, DNA-activated, catalytic polypeptide, and b) a loss of function mutation in the gene that encodes for the interleukin 2 receptor α.

Disclosed herein are transgenic mice, comprising: one or more human CD45 expressing cells, human B cells, human T cells, human dendritic cells, human monocytes/macrophages, human NK cells, human innate lymphoid cells, human microglia or human iNKT cells; and wherein the mouse's endogenous immune system is immunodeficient.

Disclosed herein are methods of making a transgenic mouse with a human immune system, the methods comprising: engrafting a mouse with human hematopoietic cells, wherein the engrafting is intracardial, wherein the mouse's endogenous immune system is immunodeficient and wherein the transgenic mouse comprises one or more human CD45 expressing cells, human B cells, human T cells, human dendritic cells, human monocytes/macrophages, human NK cells, human innate lymphoid cells, human microglia or human iNKT cells.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “comprising” can include the aspects “consisting of” and “consisting essentially of.” “Comprising can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the subject afflicted or those in contact with a subject. A disease or disorder or condition can also related to a distemper, ailing, ailment, malady, disorder, sickness, illness, complaint, affection.

As used herein, the term “normal” refers to an individual, a sample or a subject that does not have a disease or disorder.

The phrase “at least” preceding a series of elements is to be understood to refer to every element in the series. For example, “at least one” includes one, two, three, four or more.

As used herein, the term “transformation” refers to a permanent or transient genetic change induced in a cell following incorporation of exogenous DNA to the cell.

As used herein, the term “lymphocyte” includes natural killer cells, T cells, and B cells; and are the main type of cell found in the lymph. “Mature” lymphocytes can be defined by their cell surface receptor. For example, B cell receptor or immunoglobulin for B cells and T cell receptor of T cells. A mature lymphocyte can be selected based on its ability to differentiate between self and non-self.

Lymphocytes are the central cell type of the adaptive immune system, and represent 20-40% of white cells in the blood. Small lymphocytes range between 7 and 10 μm in diameter. They are characterized by a nucleus that stains dark purple with Wright's stain, and by a small cytoplasm. Large granular lymphocytes range between 10 and 12 μm in diameter and contain more cytoplasm and scattered granules.

In some aspects, when lymphocytes are stimulated by an antigen (a foreign protein on the surface of a microorganism or allergen), the B lymphocytes are transformed into plasma cells which synthesize and release antibodies (gamma globulins). As described herein, the genetically modified mice can mount a fully mature antibody response complete with the production of memory B cells and plasma cells.

The term “xenogeneic” is used herein with reference to a host cell or organism to indicate that the material referred to as “xenogeneic” is derived from another species than that of the host cell or organism.

As used herein, the term “healthy” refers to an individual or subject or a part of the body that is not diseased or is free of a disease or disorder or condition.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

### GENERAL DESCRIPTION

Disclosed herein is a mouse model that was developed by engrafting human umbilical cord blood derived hematopoietic stem cells (CD34+) intracardially into an immunodeficient NOD.Cg-Kit^{W-41J} Prkdc^{scid} I12rgtm1Wjl/WaskJ (NSGW41) or NOD.Cg-Kit^{W-41J} Tyr⁺ Prkdc^{scid} Il2rg^{tm1Wjl}/Thom0J (NBSGW) mouse. During development, β-estradiol was administered within the drinking water to promote myeloid lineage differentiation and maturation of the T and B lymphocyte compartments. Without β-estradiol, the development of the human immune system was stunted and lacked the ability to produce mature antibody responses in the immunodeficient NSGW41 and NBSGW mice.

An advantage of the genetically modified mice disclosed herein is that it supports human specific pathogens (such as chlamydia trachomatis), suppresses human tumor cell growth through the function of the human immune system, and mounts a class-switched, hypermutated and high affinity antibody response to both T-lymphocyte-dependent and independent antigens. Additionally, the mature human immune response in the disclosed genetically modified mice can be complete with robust generation of memory B cells, plasma cells, and T-lymphocyte memory.

While previously developed genetically modified mice have had limited human immune system reconstitution (20-40% within peripheral blood) with deficiencies in major cell compartments, including B-lymphocytes, the genetically modified mice described herein was developed by grafting NOD.Cg-Kit^{W-41J} Prkdc^{scic} Il3rg^{tm1Wjl}/WaskJ (NSGW41) and NOD.Cg-Kit^{W-41J} Tyr⁺ Prkdc^{scid} Il2rg^{tm1Wjl}/Thom0J (NBSGW) mice within 48 hours of birth, devoid of prior irradiation, with human hematopoietic (CD34+) stem cells. Human umbilical cord hematopoietic stem cells were collected within 30 minutes post-partum and injected intracardially, consistently yielding up to 95% human cell peripheral reconstitution. Human leukocyte development, differentiation and long-term persistence in these CD34⁺ cell-grafted NSGW41 mice revealed B and T cell maturation as part of a full immune system development, which led to the emergence of IgM, IgG, IgA and IgE antibody titers comparable to adult humans. CD34+ cell-grafted NSGW41 and NBSGW mice support specific T-dependent and T-independent antibody responses that include human B cell class switch DNA recombination, plasma cell and memory B cell differentiation. They also support a hydrocarbon-induced autoantibody response leading to symptoms modeling systemic lupus erythematosus that can be utilized for drug testing and development. Thus, disclosed herein are robust in vivo platforms allowing for generation and maturation of human antibody and autoantibody responses.

As described herein, the transgenic mice disclosed herein can be used to study drug interactions on the human immune system; to produce human polyclonal and monoclonal antibodies against any antigen; to generate hybridomas producing fully human antibodies; to study human pathogens within an in vivo human immune system rather than using a mouse adapted pathogens within inbred strains of mice; and since the human hematopoietic stem cells are derived from human umbilical cords, each litter of transgenic mice can have a full human genome. This adds value to the platform as it can be utilized in investigating the impact of diseases and therapeutics across genetic variations seen among human patients and can provide an in vivo platform for personalized medicine research.

Disclosed herein are transgenic mice, comprising: one or more human CD45 expressing cells, human B cells, human T cells, human dendritic cells, human monocytes/macrophages, human NK cells, human innate lymphoid cells, human microglia or human iNKT cells. In some aspects, the mice's endogenous immune system can be immunodeficient. In some aspects, the transgenic mice can comprise one or more mutations. In some aspects, the one or more mutations can be: a loss of function mutation causing the mode of action (moa) loss-of-function mutation in the gene that encodes for the protein kinase, DNA-activated, catalytic polypeptide; a loss-of-function mutation in the gene that encodes for the interleukin 2 receptor α; or a loss-of-function mutation in a gene that encodes for a KIT receptor. In some aspects, the transgenic mice described herein can further comprise an engraftment of human hematopoietic stem cells.

Disclosed herein are transgenic mice generated by engrafting CD34⁺ stem cells isolated by magnetic selection from human umbilical cord blood. Intracardial engraftment of mice (e.g., immunodeficient mice) can yield up to 95% human cell peripheral reconstitution and supports full human leukocyte development, differentiation and persistence beyond 1 year of age without development of xeno-reactive graft-versus-host reaction and disease.

Disclosed herein are transgenic mice that reconstitute all major human hematopoietic lineage cells and their respective subsets, including but not limited to B cells, T cells, monocytes and macrophages, dendritic cells, NK cells, iNKT cells, innate lymphoid cells and red blood cells reaching 98% human reconstitution within the bone marrow and secondary lymphoid organs, including spleen, mesenteric lymph node and gut-associated lymphoid tissues, as enhanced by estrogen administration.

Disclosed herein are transgenic mice that support human physiological development and rearrangement of both B cell and T cell receptors to generate repertoire diversity comparable to healthy adult humans which is important for producing antibodies against a broad range of antigens.

Disclosed herein are transgenic mice that support B and T cell maturation as part of a full immune system development, which leads to emergence of IgM, IgD, IgG, IgA and IgE antibody titers comparable to those in adult humans and reconstitutes all major hematopoietic lineage compartments, as enhanced by estrogen administration.

Disclosed herein are transgenic mice with expanded myeloid lineage and T lymphocyte compartments, T memory cell generation, including mucosal sites, such as the lungs, as enhanced by estrogen administration.

Disclosed herein are transgenic mice with dynamically increased antibody class-switch DNA recombination, AID and BLIMP1 expression, B memory cell generation and plasma cell differentiation, as potentiated by estrogen administration.

Disclosed herein are transgenic mice that support human B cell development and differentiation to the extent that B cells undergo antibody class-switch DNA recombination and plasma cell differentiation in response to in vitro stimulation as efficiently as B cells isolated from healthy adult donors.

Disclosed herein are transgenic mice that support in vivo induction and maturation of T lymphocyte-dependent and T lymphocyte-independent antibody responses, including antibody class-switch DNA recombination, somatic hypermutation, plasma cell differentiation and memory B cell differentiation as well as development of peripheral germinal center or germinal center-like structures or secondary lymphoid organizations, as boosted by estrogen administration.

Disclosed herein are transgenic mice in which maturation of such T lymphocyte-dependent and T-independent antibody responses is vastly potentiated by estrogen administration.

Disclosed herein are transgenic mice that support systemic autoantibody responses induced by pristane or other agents leading to systemic or organ-specific autoimmunity. In some aspects, pristane can induce a systemic lupus erythematosus-like disease complete with IgM, IgG, IgA and IgE autoantibodies, autoimmune pathology including but not limited to deposition of autoantibodies in kidneys and glomerulonephritis, eventually leading to glomerulosclerosis in the transgenic mice described herein.

Disclosed herein are transgenic mice that support the induction of IgE-mediated hypersensitivity to yield allergic responses to respiratory and alimentary allergens, including but not limited to house-dust mite and peanuts, as facilitated and enhanced by estrogen administration.

Disclosed herein are transgenic mice that can be induced to develop or support engraftment and rejection of liquid and solid tumors as to be adapted as models for identification of therapeutic targets.

Disclosed herein are transgenic mice that support vaccine and therapeutic development through testable predictions of the efficacy of immunogens to identify and target defined lymphocyte subsets expressing antigen receptors capable of inducing protective humoral immune responses.

In contrast to other transgenic mouse models, the disclosed transgenic mice can undergo antibody class-switch DNA recombination, somatic hypermutation, plasma cell differentiation and memory B cell differentiation.

Disclosed herein are transgenic mice that can allow for generation of fully human monoclonal antibodies and generation of hybridomas of predetermined isotype and specificity.

Disclosed herein are transgenic mice that can produce fully human antibodies with human secondary modifications, including but not limited to glycosylation of antibody constant regions, with the ability to modulate antibody effector function, immunogenicity and half-life, as facilitated and enhanced by estrogen administration.

Disclosed herein are transgenic mice that can support human-specific infections due to the reconstitution of human cells.

Disclosed herein are transgenic mice that can support therapeutic development and transplantation advances through approaches including but not limited to de-risking of biological therapeutics, personalized medicine diagnostic methodologies due to each mouse containing a full human-genome, testable predictions of therapeutic interactions with the human immune system, immunotherapy toxicity and investigation of allograft rejection.

Disclosed herein are transgenic mice that can be co-grafted with human non-hematopoietic stem cell progenitors, for example, those obtained from human cord vascular lining cells, including but not limited to lymphoid tissue organizer cells that give rise to lymphoid tissue inducer cells, marginal reticular cells, follicular dendritic cells and fibroblastic reticular cell precursors leading to improved secondary lymphoid development and human reconstitution through generating a human microenvironment.

Disclose herein are transgenic mice comprising: a functional human immune system; and one or more human hematopoietic stem cells. Disclosed herein are transgenic mice, comprising: one or more human CD45 expressing cells, human B cells, human T cells, human dendritic cells, human monocytes/macrophages, human NK cells, human innate lymphoid cells, human microglia or human iNKT cells; and wherein the mouse's endogenous immune system is immunodeficient. Further disclosed herein are genetically modified mice comprising: a) a loss-of-function mutation in the gene that encodes for the protein kinase, DNA-activated, catalytic polypeptide; and b) a loss-of-function mutation in the gene that encodes for the interleukin 2 receptor α; wherein the mouse further comprises an engraftment of human hematopoietic stem cells. In some aspects, the engraftment of the human hematopoietic stem cells can be through an intracardial injection. As used herein, the transgenic mice described herein can also be referred to as “the humanized mouse”, “H-Mouse”, “H-Mice”, “huMouse”, “huMice”, “NSGW41 H-Mouse”, “NSGW41 H-Mice”, “NBSGW H-Mouse”, “NBSGW H Mice”, “NGS H-Mouse”, “NGS-H Mice”, or “immunocompetent mouse” or “immunocompetent genetically modified mouse.” In some aspects, the transgenic mice described herein with a human immune system can be described by the background used to generate the humanized mouse. A “background mouse” as used herein refers to an immunocompromised mouse and can be referred to as the “immunodeficient mouse” or “immunodeficient genetically modified mouse”. In some aspects, the background mouse can be NGS, NSGW41 or NBSGW.

In some aspects, the transgenic mice described herein can comprise one or more mutations. In some aspects, the one or more mutations can be: a loss of function mutation causing the moa loss-of-function mutation in the gene that encodes for the protein kinase, DNA-activated, catalytic polypeptide; a loss-of-function mutation in the gene that encodes for the interleukin 2 receptor α; or a loss-of-function mutation in a gene that encodes for a KIT receptor. In some aspects, the transgenic mice described herein can further comprise an engraftment of human hematopoietic stem cells.

In some aspects, in any of the transgenic mice described herein, can further comprise genetically editing one or more human genes prior to the engraftment of the human hematopoietc stem cells. In some aspects, the human hematopoietc stem cells can comprise an insertion, a deletion or a modification of one or more human genes prior to the engraftment step.

In some aspects, the transgenic mice described herein comprise NOD.Cg-Kit^{W-41J} Prkdc^{scid} Il2rg^{tm1Wjl}/WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/S_{z}J (NSG). Mice comprising NOD.Cg-Kit^{W-41J} Prkdc^{scid} Il2rg^{tm1Wjl}/WaskJ (NSGW41), NOD.Cg-Kit^{W-41J} Tyr⁺Prkdc^{scid} Il2rg^{tm1Wjl}/Thom0J (NBSGW) or NOD.Cg-Prkdc^{scid}Il2rg^{tm1Wjl}/SzJ (NSG) can be used a background mice in the methods described herein as they are examples of immunodeficient mouse (i.e., mice whose endogenous immune system is immunodeficient).

In some aspects, the mice disclosed herein can be treated with an estrogen receptor agonist. In some aspects, the mice disclosed herein can be treated with estrogen or estradiol.

In some aspects, the transgenic mice disclosed herein comprises functional human immune system. As used herein, the term “functional human immune system” can include one or more or all of the human hematopoietic stem cell lineage cells. The term “functional human immune system” as used herein can refer to a mouse that comprises B and T cell maturation as part of a full immune system development, which leads to emergence of IgM, IgD, IgG, IgA and IgE antibody titers comparable to those in adult humans; T lymphocyte-dependent and T lymphocyte-independent antibody responses, including antibody class-switch DNA recombination, somatic hypermutation, plasma cell differentiation and memory B cell differentiation as well as development of peripheral germinal center or germinal center-like structures or secondary lymphoid organizations; a human immune system that supports the induction of IgE-mediated hypersensitivity to yield allergic responses to respiratory and alimentary allergens; and a human immune system that can be induced to develop or support engraftment and rejection of liquid and solid tumors.

In some aspects, the transgenic mice described herein can comprise mature human leukocytes. In some aspects, the immunocompetent genetically modified mouse can comprises one or more human hematopoietic lineage cells. In some aspects, the one or more human hematopoietic lineage cells is a B cell, a T cell, a monocyte, a macrophage, a dendritic cell, a NK cell, a iNKT cell, an innate lymphoid cell, a microglia or a red blood cell. In some aspects, the disclosed transgenic mice comprises all human hematopoietic lineage cells. In some aspects, the one or more human hematopoietic lineage cells can be maintained up to 40 weeks. In some aspects, the one or more hematopoietic lineage cells can be maintained 40 weeks or longer. In some aspects, the one or more hematopoietic lineage cells can be maintained 50 weeks or longer. In some aspects, the one or more hematopoietic lineage cells can be maintained 1 year, 1.5 years or 2 years or any amount of time in between. In some aspects, the one or more human hematopoietic lineage cells can reach 98% human reconstitution. In some aspects, the intracardial engrafted hematopoietic stem cells can reach up to 95% human cell peripheral reconstitution.

In some aspects, the engrafted hematopoietic stem cells are capable of developing into one or more of a human B cell, a human T cell, a human monocyte, a human macrophage, a human dendritic cell, a human NK cell, a human iNKT cell, a human innate lymphoid cell, a human microglia and a human red blood cell or a combination thereof. In some aspects, any of the human B cell, a human T cell, a human monocyte, a human macrophage, a human dendritic cell, a human NK cell, a human iNKT cell, a human innate lymphoid cell, a human microglia and a human red blood cell or a combination thereof can look different than mouse cells based off morphology, gene expression, intra- and extracellular protein expression, etc.

In some aspects, the transgenic mice described herein can comprise at least one of each of a human B cell, a human T cell, a human monocyte, a human macrophage, a human dendritic cell, a human NK cell, a human iNKT cell, a human innate lymphoid cell, a human microglia and a human red blood cell. In some aspects, the at least one of each of a human B cell, a human T cell, a human monocyte, a human macrophage, a human dendritic cell, a human NK cell, a human iNKT cell, a human innate lymphoid cell, a human microglia and a human red blood cell can reach at least 95% human reconstitution within bone marrow or at least 98% within a secondary lymphoid organ in response to estrogen stimulation. In some aspects, the secondary lymphoid organ can be a spleen, a mesenteric lymph node or a gut-associated lymphoid tissue. In some aspects, the transgenic mice described herein has not been irradiated.

In some aspects, the transgenic mice described herein is capable of producing one or more antibodies. In some aspects, the one or more antibodies produced can support the development of one or more immunoconjugates. In some aspects, the one or more immunoconjugates can be used for research. In some aspects, the one or more immunoconjugates can be used as a therapy to treat one or more human diseases, disorders or conditions.

In some aspects, the transgenic mice described herein is capable of physiological development and rearrangement of human B cell and T cell receptors thereby generating a repertoire diversity comparable to a healthy adult human for producing one or more human antibodies against a broad range of antigens. As used herein, the phrase “T cell receptor repertoire” or “T cell receptor profile” refers to the sum of all the human T cell receptors by the human T cells of an individual. The T cell receptor repertoire can change with the onset and progression of diseases. The term “diverse” as it relates to phrase “diverse T cell receptor repertoire” means the T cells undergo gene rearrangement during development to form a wide array of T cell receptors in which a pool of T cells is capable of recognizing a diverse set of antigens. This also applies to the B cell repertoire and implies that the development of human T cells and B cells recapitulates normal development occurring within humans. In some aspects, the transgenic mice described herein is capable of producing human IgM, IgD, IgG (e.g., IgG1, IgG2, IgG3 and IgG4), IgA (e.g., IgA1 and IgA2) or IgE antibodies or antibody titers. In some aspects, the human IgM, IgD, IgG (e.g., IgG1, IgG2, IgG3 and IgG4), IgA (e.g., IgA1 and IgA2) or IgE antibody titers are comparable to those in an adult human in response to estrogen stimulation or estrogen conditioning.

In some aspects, the transgenic mice described herein comprises or is capable of producing an expanded myeloid lineage and T lymphocyte compartments. In some aspects, the expansion of myeloid lineage and T lymphocyte compartments can be potentiated by estrogen administration.

In some aspects, the transgenic mice described herein comprises or is capable of producing one or more human T memory cells.

In some aspects, the transgenic mice described herein comprise or are capable of producing human immune system reconstitution of one or more mucosal sites. In some aspects, the transgenic mice described herein comprise or are capable of producing human immune cells within on or more mucosal sites. In some aspects, the one or more mucosal sites is in the lungs.

In some aspects, wherein the transgenic mice described herein comprise or are capable of undergoing an increased AID and BLIMP1 expression, antibody class-switch DNA recombination, affinity maturation, somatic hypermutation, and/or B memory cell generation and plasma cell differentiation in response to estrogen stimulation.

In some aspects, the transgenic mice described herein are capable of supporting human B cell development and differentiation to the extent that B cells express AID and BLIMP1, undergo antibody class-switch DNA recombination and plasma cell differentiation in response to in vitro stimulation as efficiently as B cells isolated from a healthy adult donor.

In some aspects, the transgenic mice described herein provide a renewable source of one or more human hematopoietic lineage cells. In some aspects, the one or more human hematopoietic lineage cells include one or more of a human B cell, a human T cell, a human monocyte, a human macrophage, a human dendritic cell, a human NK cell, a human iNKT cell, a human innate lymphoid cell, a human microglia and a human red blood cell or a combination thereof.

In some aspects, the transgenic mice described herein are capable of supporting in vivo induction and maturation of a T lymphocyte-dependent or a T lymphocyte-independent antibody response. In some aspects, the T lymphocyte-dependent or a T lymphocyte-independent antibody response can be potentiated in response to estrogen administration. In some aspects, the antibody response has undergone one or more of an antibody class-switch DNA recombination, a somatic hypermutation, a plasma cell differentiation, a memory B cell differentiation, development of peripheral germinal center or germinal center-like structures or secondary lymphoid organizations.

In some aspects, the transgenic mice described herein are capable of supporting a systemic autoantibody response, wherein the systemic autoantibody response is induced by pristane thereby resulting in systemic or organ-specific autoimmunity. In some aspects, the systemic or organ-specific autoimmunity can be a systemic lupus erythematosus-like disease. In some aspects, the systemic lupus erythematosus-like disease comprises IgM, IgG, IgA and IgE autoantibodies. In some aspects, the one or more IgM, IgG, IgA and IgE autoantibodies can be present in a kidney or glomerulonephritis.

In some aspects, the transgenic mice described herein are capable of supporting induction of IgE-mediated hypersensitivity. In some aspects, the IgE-mediated hypersensitivity can yield an allergic response to a respiratory or an alimentary allergen. In some aspects, the respiratory or the alimentary allergen can be a house-dust mite, a peanut or other respiratory tract allergens. In some aspects, the IgE-mediated hypersensitivity can be facilitated by estrogen administration. In some aspects, the IgE-mediated hypersensitivity can be boosted by estrogen administration.

In some aspects, the transgenic mice described herein are capable of being induced to develop or support engraftment and rejection of a liquid or a solid tumor including patient-derived xenograft.

In some aspects, the transgenic mice described herein are capable of supporting vaccine and therapeutic development through testing the efficacy of an immunogen to identify and target a defined lymphocyte subset. In some aspects, the defined lymphocyte subset can express an antigen receptor capable of inducing a protective humoral immune response.

In some aspects, the transgenic mouse described herein are capable of supporting development of one or more therapeutics for one or more autoimmune diseases or allergic diseases through testing the efficacy of a small molecule compound to identify and target defined lymphocyte surface, intracellular molecules or different cell subsets.

In some aspects, the transgenic mice described herein are capable of generating a fully human monoclonal antibody. In some aspects, one or more B lymphocytes can be isolated from the transgenic mice described herein. In some aspects, the one or more isolated B lymphocytes can be used to generate a human hybridoma of a predetermined antibody isotype and specificity. In some aspects, the transgenic mouse is capable of generating a human hybridoma of a predetermined antibody isotype and specificity.

In some aspects, the transgenic mice described herein are capable of supporting one or more human-specific infections. In some aspects, the transgenic mice described herein are capable of supporting one or more human microbial infections.

In some aspects, the transgenic mice described herein can comprise 95% human cell peripheral reconstitution.

In some aspects, the transgenic mice described herein are capable of supporting full human leukocyte development, differentiation and persistence beyond 1 year of age without developing xeno-reactive graft-versus-host reaction and disease.

Estrogen. In some aspects, the transgenic mice described herein can be treated with an estrogen receptor agonist. In some aspects, the transgenic mice described herein can be treated with estrogen or estradiol. As described herein, the administration of estrogen can potentiate one or more of the outcomes of a functional human immune system in the transgenic mice described herein, however, the stimulation is not due to estrogen. Stimulation of the human cells is by antigens, and the estrogen acts through one or more other mechanisms to boost (potentiate or enhance) the response.

Immunodeficient Mice. In some aspects, the transgenic mice described herein are generated from an immunodeficient mouse. As described herein, the transgenic mice described herein can be produced from an immunodeficient mouse that is the recipient of the human hematopoietic stem cells. The transgenic mice described herein and/or an immunodeficient mouse can contain a genetically modified endogenous gene or chromosomal locus such that the mouse does not have a functioning native immune system. In some aspects, the transgenic mice described herein and/or an immunodeficient mouse does not express a functional DNA-activated, catalytic polypeptide. In some aspects, the transgenic mice described herein and/or an immunodeficient mouse does not express a functional interleukin 2 receptor α. In some aspects, the transgenic mice described herein and/or an immunodeficient mouse can comprise the strain NOD.Cg-Kit^{W-41J} Prkdc^{scid} Il2rg^{tm1Wjl}/WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

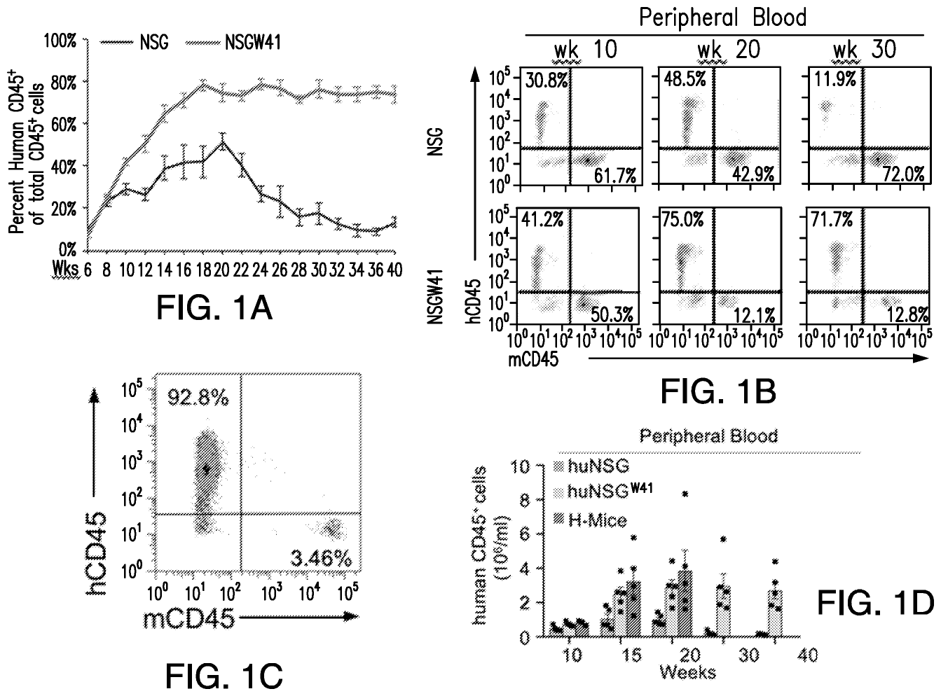

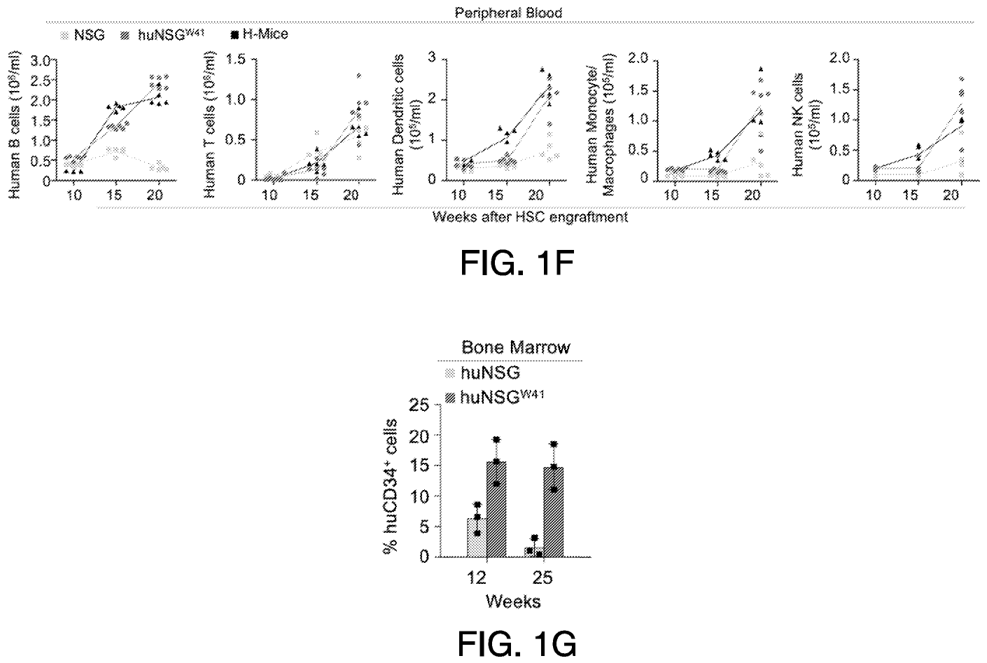

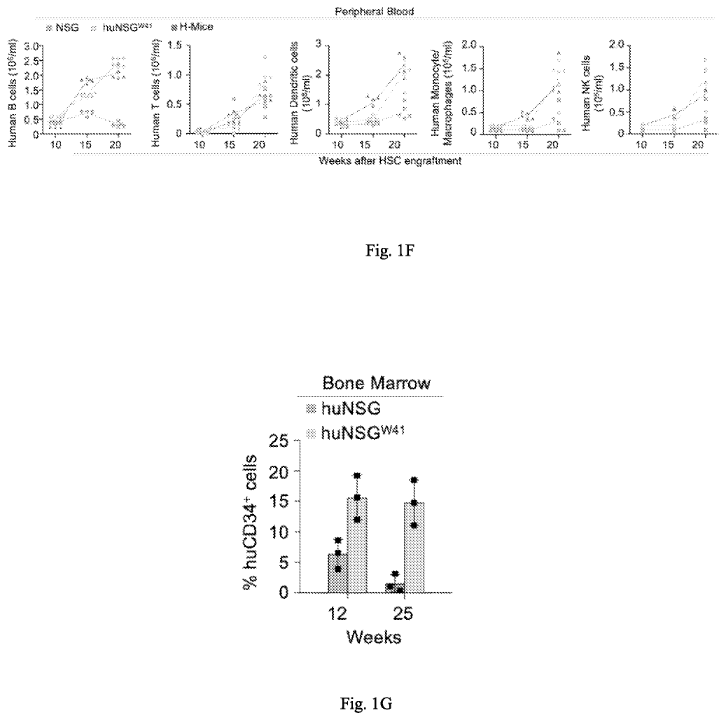

In some aspects, a transgenic mice described herein and/or an immunodeficient mouse lacking a kit mutation (e.g., NOD.Cg-Prkdc^{scid}Il2rg^{tm1Wjl}/SzJ (NSG)) may require irradiation prior to engraftment with human HSCs as mice lacking a kit mutation support a 50% human reconstitution (see, for example, FIG. 1A) compared to 80% human reconstitution achieved in mouse strains having a kit mutation.

In some aspects, a transgenic mice described herein and/or an immunodeficient mouse can further comprise a loss-of-function mutation in a gene that encodes for a KIT receptor. In some aspects, a transgenic mice described herein and/or an immunodeficient mouse does not express a functional KIT receptor. In some aspects, the loss-of-function mutation in the gene that encodes for the protein kinase, DNA-activated, catalytic polypeptide comprises an T-to-A transversion point mutation at a position corresponding to codon 4046 (codon 4095 in transcript ENSMUST00000023352.8; creating a premature stop codon). In some aspects, the loss-of-function mutation in the gene that encodes for the protein kinase, DNA-activated, catalytic polypeptide is Prkdc^{scid}. In some aspects, the loss-of-function mutation in the gene that encodes for the interleukin 2 receptor α comprises a neomycin resistance cassette (e.g., the neomycin resistance cassette that replaced part of exon 3 and exons 4-8 of the gene, resulting in the loss of most of the extracellular domain and all of the transmembrane and cytoplasmic domains of the protein). In some aspects, the loss-of-function mutation in the gene that encodes for the interleukin 2 receptor α is Il2rg^{tm1Wjl}. In some aspects, the loss-of-function mutation in the gene that encodes for the KIT receptor comprises a G to A point mutation in the kinase domain at nucleotide 2519. Said point mutation results in a valine to methionine substitution at amino acid 831. In some aspects, the loss-of-function mutation in the gene that encodes for the KIT receptor is Cg-Kit^{W-41J}. In some aspects, the immunodeficient genetically modified mouse comprises NOD.Cg-Kit^{W-41J} Prkdc^{scid} Il2rg^{tm1Wjl}/WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

In some aspects, the transgenic mice described herein and/or an immunodeficient mouse has not been irradiated.

“Hematopoietic stem cells” or “HSCs” as used herein refers to primitive cells capable of regenerating all blood cells. During development, the site of hematopoiesis translocates from the fetal liver to the bone marrow, which then remains the site of hematopoiesis throughout adulthood. HSCs as used herein refers to pluripotent stem cells or multipotent stem cells or lymphoid or myeloid (derived from bone marrow) stem cells that, upon exposure to an appropriate cytokine or plurality of cytokines, can either differentiate into a progenitor cell of a lymphoid, erythroid, or myeloid cell lineage or proliferate as a stem cell population without further differentiation being initiated. HSCs can be isolated from bone marrow, peripheral blood, umbilical cord blood, or embryonic stem cells. HSCs can form cells such as erythrocytes (e.g., reticulocytes, erythrocytes), thrombocytes (e.g., megakaryoblasts, platelet producing megakaryocytes, platelets), granulocytes (e.g., promyelocytes, neutrophils, eosinophils, basophils)), monocytes (e.g., monocytes, macrophages), and/or lymphocytes (e.g., B cells, T cells, natural killer cells). HSC are capable of self-renewal or remaining a stem cell after cell division. HSCs are also capable of differentiation or starting a path to becoming a mature hematopoietic cell. HSCs can also be regulated in their mobility or migration or can be regulated by apoptosis or programmed cell death.

In some aspects, the human hematopoietic stem cells can comprise one or more cells selected from the group consisting of a human CD34-positive cell, a human hematopoietic stem cell, a human myeloid precursor cell, a human erythroid precursor cell, a human myeloid cell, a human dendritic cell, a human monocyte, a human granulocyte, a human erythrocyte, a human neutrophil, a human mast cell, a human thymocyte, and a human B lymphocyte. In some aspects, the hematopoietic stem cells can be CD34+ stem cells.

HSCs may or may not include CD34+ cells. CD34+ cells are immature cells that express the CD34 cell surface marker. CD34+ cells are believed to include a subpopulation of cells with the stem cell properties described herein. HSCs include pluripotent stem cells, multipotent stem cells (e.g., a lymphoid stem cell), and/or stem cells committed to specific hematopoietic lineages. The stem cells committed to specific hematopoietic lineages can be of T cell lineage, B cell lineage, dendritic cell lineage, Langerhans cell lineage and/or lymphoid tissue-specific macrophage cell lineage. In addition, HSCs also refer to long-term HSC (LT-HSC) and short-term HSC (ST-HSC). A long-term stem cell typically includes the long-term (more than three months) contribution to multilineage engraftment after transplantation. A short-term stem cell is typically anything that lasts shorter than three months, and/or that is not multilineage. LT-HSC and ST-HSC are distinguished, for example, based on their cell Surface marker expression. LT-HSC are CD34−, SCA-1+, Thy 1.1+/lo, C-kit+, Un-CD135−, Slamfl/CD150+, whereas ST-HSC are CD34+, SCA-1+, Thy 1.1+/lo, C-kit+, lin−, CD135−, Slamfl/CD150+, Mac-1 (CD1 Ib)lo (Handbook of Stem Cells, 2004). In addition, ST-HSC are less quiescent (i.e., more active) and more proliferative than LT-HSC. LT-HSC have unlimited self-renewal (i.e., they survive throughout adulthood), whereas ST-HSC have limited self-renewal (i.e., they survive for only a limited period of time). In some aspects, the hematopoietic stem cells can be CD34+ stem cells.

In some aspects, the transgenic mice described herein can comprise one or more human CD45 expressing cells, human B cells, human T cells, human dendritic cells, human monocytes/macrophages, human NK cells, human innate lymphoid cells, human microglia, human iNKT cells or a combination thereof. In some aspects, the transgenic mice described herein can have an increased percentage and number of human immune cells as compared to an existing mouse model (e.g., NSG).

Examples of mice that can be compared to the transgenic mice described herein (e.g., humanized mouse) are described in: Blood, 2017 Feb. 23; 129(8): 959-969, Cell Stem Cell., 2014 Aug. 7; 15(2):227-38, Stem Cell Reports, 2015 Feb. 10; 4(2): 171-180, Blood. 2005 Sep. 1; 106(5):1565-73, U.S. Pat. Nos. 9,668,463, and 10,378.038; these references are hereby incorporated herein by reference. Mice described in these references have been shown to have lower percentages of human immune cells, fewer total cell numbers within the peripheral blood, and the human reconstitution is not supported for the same duration compared to the humanized mice disclosed herein. For instance, the transgenic mice described herein can maintain an average of 75-85% (with some mice reaching 95%) human immune cells within the peripheral blood, 98% within the spleen and lymph nodes, and >95% within the bone marrow. The human reconstitution in the transgenic mice described herein is supported through 40 weeks compared to a loss of human cells starting at 12-20 weeks post-engraftment in these previous studies. As described herein, the peripheral blood supports absolute numbers of >4*10⁶ human CD45⁺ cells per ml. 2.25-2.75*10⁶ B cells per ml, 1.5-2*10⁶ T cells per ml, 2-2.5*10⁵ dendritic cells per ml, 1-2*10⁵ monocyte/macrophage cells per ml, 1-2*10⁵ NK cells per ml.

The terms “progenitor and “progenitor cell” as used herein refer to primitive hematopoietic cells that have differentiated to a developmental stage that, when the cells are further exposed to an appropriate cytokine or a group of cytokines, they will differentiate further along the hematopoietic cell lineage. In contrast to HSCs, progenitors are only capable of limited self-renewal and are not capable of long-term self-renewal. Thus, hematopoietic progenitor cells can restore and sustain hematopoiesis for three to four months (Marshak et al., 2001) and are important for recovery in the period immediately following a hematopoietic progenitor cell transplant in an individual.

“Progenitors” and “progenitor cells” as used herein also include “precursor cells that are derived from differentiation of progenitor cells and are the immediate precursors of mature differentiated hematopoietic cells. The terms “progenitor” and “progenitor cell” as used herein include, but are not limited to, granulocyte-macrophage colony-forming cell (GM-CFC), megakaryocyte colony-forming cell (MK-CFC), burst-forming unit erythroid (BFU-E), B-cell colony-forming cell (B-CFC), and T-cell colony-forming cell (TCFC). “Precursor cells” include, but are not limited to, colony forming unit-erythroid (CFU-E), granulocyte colony-forming cell (G-CFC), colony-forming cell-basophil (CFC-Bas), colony-forming cell-eosinophil (CFC-Eo), and macrophage colony-forming cell (M-CFC) cells.

In some aspects, the transgenic mice described herein can further comprise one or more human non-hematopoietic stem cell progenitors. In some aspects, the transgenic mice described herein further comprises an engraftment of one or more human non-hematopoietic stem cell progenitors. In some aspects, the transgenic mice described herein can also further comprises a co-engraftment of human CD34-positive cells with one or more human non-hematopoietic stem cell progenitors. In some aspects, the one or more human non-hematopoietic stem cell progenitors can be obtained from human cord. In some aspects, the one or more human non-hematopoietic stem cell progenitors can be obtained from vascular lining cells, Wharton's jelly and/or perivascular tissue. Examples of human non-hematopoietic stem cell progenitors include but are not limited to lymphoid tissue organizer cells (lymphoid tissue organizer cells differentiate into lymphoid tissue inducer cells), marginal reticular cells, follicular dendritic cells and fibroblastic reticular cell precursors. In some aspects, the one or more human non-hematopoietic stem cell progenitors can be lymphoid tissue organizer cells, lymphoid tissue inducer cells, marginal reticular cells, follicular dendritic cells or fibroblastic reticular cell precursors. In some aspects, the one or more human non-hematopoietic stem cell progenitors are capable of improving secondary lymphoid development and structure; forming a human microenvironment within the secondary lymphoid; increasing the recruitment of human lymphocytes to secondary lymphatic tissues through human cytokine and chemokine production promoting chemotaxis; improving the development of the peripheral germinal center or germinal center-like structures or secondary lymphoid organizations; increasing human cytokine production, including human IL-6 and human BAFF; increasing antigen presentation by human cells; and improving T-lymphocyte dependent and T-lymphocyte independent antibody response.

As described herein, the transgenic mice disclosed herein can be compared to any of the mouse models described in Blood, 2017 Feb. 23; 129(8): 959-969, Cell Stem Cell., 2014 Aug. 7; 15(2):227-38, Stem Cell Reports, 2015 Feb. 10; 4(2): 171-180, Blood. 2005 Sep. 1; 106(5):1565-73, U.S. Pat. Nos. 9,668,463, and 10,378,038. In these mouse models lymphatic tissue development is deficient.

Methods of Making

The NOD.Cg-Prkdc^{scid}Il2rg^{tm1Wjl}/SzJ (NSG) mouse strain (lacking a kit mutation) requires irradiation prior to engraftment with human HSCs and supports only 50% human reconstitution (see, for example FIG. 1A) compared to 80% achieved in mouse strains having the kit mutation. Therefore, the methods of making a mouse with a human immune system (e.g. a transgenic mice described herein), and in particular, the step of engrafting described herein achieves greater, more sustained human engraftment than previous studies can be conducted on a mouse strain carrying a kit mutation (e.g., an immunodeficient mouse strain carrying kit mutation(s)).

The advantages of the transgenic mice described herein include but are not limited to: having no observable development of xeno-reactive graft-versus-host reaction and disease; mice that live up to 1.5 years (and having no significant difference in lifespan compared to same strain of mice receiving no human cells); estrogen administration leads to the expansion of the T follicular helper cell compartment; estrogen administration leads to an expansion of myeloid lineage cells; and estrogen administration leads to an expansion of NK cells.

Disclosed herein are methods of making a transgenic mouse with a human immune system. In some aspects, the methods comprise: engrafting a mouse with human hematopoietic cells. In some aspects, the engrafting can be intracardial. In some aspects, the mouse's endogenous immune system can be immunodeficient. In some aspects, the transgenic mouse can comprise one or more human CD45 expressing cells, human B cells, human T cells, human dendritic cells, human monocytes/macrophages, human NK cells, human innate lymphoid cells, human microglia or human iNKT cells. In some aspects, the engrafted mouse comprises one or more mutations. In some aspects, the one or more mutations can be: a loss of function mutation causing the moa loss-of-function mutation in the gene that encodes for the protein kinase, DNA-activated, catalytic polypeptide; a loss-of-function mutation in the gene that encodes for the interleukin 2 receptor α; or a loss-of-function mutation in a gene that encodes for a KIT receptor.

Disclosed herein are methods of making a transgenic mouse with a human immune system. In some aspects, the methods comprise: engrafting a mouse with human hematopoietic cells. In some aspects, the engrafting can be intracardial. In some aspects, the mouse engrafted with the human hematopoietic cells is an immunodeficient genetically modified mouse. In some aspects, the mouse with the human immune system can be referred to as an “immunocompetent genetically modified mouse” or a transgenic mouse described herein. In some aspects, the mouse engrafted with the human hematopoietic cells (e.g. a transgenic mouse described herein) comprises: a) a loss-of-function mutation in the gene that encodes for the protein kinase, DNA-activated, catalytic polypeptide, and b) a loss of function mutation in the gene that encodes for the interleukin 2 receptor α. In some aspects, the loss-of-function mutation in the gene that encodes for the protein kinase, DNA-activated, catalytic polypeptide comprises an T-to-A transversion point mutation. The T-to-A transversion point mutation can be at a position corresponding to codon 4046 (codon 4095 in transcript ENSMUST00000023352.8) creating a premature stop codon. In some aspects, the loss-of-function mutation in the gene that encodes for the protein kinase, DNA-activated, catalytic polypeptide can be Prkdc^{scid}. In some aspects, the loss-of-function mutation in the gene that encodes for the interleukin 2 receptor α can comprise a neomycin resistance cassette. The neomycin resistance cassette replaced part of exon 3 and exons 4-8 of the gene, resulting in the loss of most of the extracellular domain and all of the transmembrane and cytoplasmic domains of the protein. In some aspects, the loss-of-function mutation in the gene that encodes for the interleukin 2 receptor α can be Il2rg^{tm1Wjl}. In some aspects, the immunodeficient genetically modified mouse can comprise NOD.Cg-Kit^{W-41J} Prkdc^{scid} Il2rg^{tm1Wjl}WaskJ (NSGW41), NOD.Cg-Kit^{W41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

Also disclosed herein are methods of making a transgenic mouse comprising a functional human immune system. In some aspects, the methods can comprise: injecting an immunodeficient genetically modified mouse with one or more human hematopoietic stem cells. In some aspects, the immunodeficient genetically modified mouse does not comprise a functional murine immune system. In some aspects, the methods can further comprise administering estrogen or estradiol to the transgenic mouse. In some aspects, the immunodeficient mouse carries or comprises the strain NOD.Cg-Kit^{W-41J} Prkdc^{scid} Il2rg^{tm1Wjl}/WaskJ mouse (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG mouse). In some aspects, the one or more human hematopoietic stem cells can be CD34+ stem cells. In some aspects, the one or more human hematopoietic stem cells can be from human umbilical cord blood. In some aspects, the transgenic mouse can comprise one or more hematopoietic lineage cells. In some aspects, the one or more hematopoietic lineage cells can be maintained up to 40 weeks. In some aspects, the one or more hematopoietic lineage cells can be maintained 40 weeks or longer. In some aspects, the one or more hematopoietic lineage cells can be maintained 50 weeks or longer. In some aspects, the one or more hematopoietic lineage cells can be maintained 1 year, 1.5 years or 2 years or any amount of time in between.

In some aspects, the human hematopoietic cells have a purity of at least 95%. In some aspects, the human hematopoietic cells have a purity of 95% or higher. In some aspects, the human hematopoietic cells have a purity of 98% or at least 98%. In some aspects, the human hematopoietic stem cells can comprise one or more cells selected from the group consisting of a human CD34-positive cell, a human hematopoietic stem cell, a human myeloid precursor cell, a human erythroid precursor cell, a human myeloid cell, a human dendritic cell, a human monocyte, a human granulocyte, a human erythrocyte, a human neutrophil, a human mast cell, a human thymocyte, and a human B lymphocyte. In some aspects, the hematopoietic stem cells can be CD34+ stem cells.

In some aspects, the method can further comprise engrafting one or more human non-hematopoietic stem cell progenitors. In some aspects, the one or more human non-hematopoietic stem cell progenitors can be lymphoid tissue organizer cells, marginal reticular cells, follicular dendritic cells, fibroblastic reticular cell precursors or a combination thereof. In some aspects, the immunodeficient genetically modified mouse does not express a functional DNA-activated, catalytic polypeptide. In some aspects, the immunodeficient genetically modified mouse does not express a functional interleukin 2 receptor α. In some aspects, the immunodeficient genetically modified mouse carries or comprises the strain NOD.Cg-Kit^{W-41J} Prkdc^{scid} Il2rg^{tm1Wjl}/WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

In some aspects, the immunocompetent genetically modified mouse can be treated with (or administered) estrogen or estradiol between day 7 and day 21. As described herein, immunocompetent genetically modified have been treated with estrogen immediately following weaning (e.g., 21 days of age) and between 1-3 weeks prior to immunization. Treatment at both time points increased functionality (e.g., generating mice having all HSC lineage cells; as defined and described herein). Estrogen treatment immediately following weaning is associated with a further increase in secondary lymphoid organ development, myeloid lineage differentiation, expanded T follicular helper compartment, and the generation of both T lymphocyte and B lymphocyte memory compared to the mice described in Cell Stem Cell., 2014 Aug. 7; 15(2):227-38, and Stem Cell Reports, 2015 Feb. 10; 4(2): 171-180.

In some aspects, estrogen can be administered orally (e.g., through drinking water) or via implantation of a slow release estrogen pellet. Both routes of administration and compositions stimulate, enhance or potentiate the percentage and number of HSC lineage cells. In some aspects, estrogen can be administered to the immunocompetent genetically modified mouse between 1 and 40 weeks. In some aspects, the concentration of estrogen can range from 0.1 μM up to lethal doses.

In some aspects, the transgenic mice described herein can comprise one or more human CD45 expressing cells, human B cells, human T cells, human dendritic cells, human monocytes/macrophages and human NK cells, human innate lymphoid cells, human microglia and human iNKT cells.

In some aspects, the transgenic mice described herein made by the methods disclosed herein has an increased percentage and absolute number of human immune cells as compared to any of the mice disclosed in Blood, 2017 Feb. 23; 129(8): 959-969, Cell Stem Cell., 2014 Aug. 7; 15(2):227-38, Stem Cell Reports, 2015 Feb. 10; 4(2): 171-180, Blood. 2005 Sep. 1; 106(5):1565-73, U.S. Pat. Nos. 9,668,463, and 10,378,038. In some aspects, the transgenic mice described herein can further comprise a loss-of-function mutation in a gene that encodes for a KIT receptor. In some aspects, the immunodeficient genetically modified mouse does not express a functional KIT receptor. In some aspects, the loss-of-function mutation in the gene that encodes for the KIT receptor can comprise a G to A point mutation in the kinase domain. The G to A point mutation in the kinase domain is at nucleotide 2519 and results in a valine to methionine substitution at amino acid 831. In some aspects, the loss-of-function mutation in the gene that encodes for the KIT receptor can be Cg-Kit^{W-41J}.

In some aspects, wherein the transgenic mice described herein has a functional human immune system. In some aspects, the transgenic mice described herein can comprise mature human leukocytes. In some aspects, the transgenic mice described herein can comprise one or more human hematopoietic lineage cells. In some aspects, the one or more human hematopoietic lineage cells can be a B cell, a T cell, a monocyte, a macrophage, a dendritic cell, a NK cell, a iNKT cell, an innate lymphoid cell, microglia or a red blood cell. In some aspects, the transgenic mice described herein comprises all human hematopoietic lineage cells. In some aspects, the one or more human hematopoietic lineage cells can be maintained for up to 40 weeks or at least 40 weeks.

Methods of Using

Disclosed herein are methods of using any of the transgenic mice described herein or any of the mice with a human immune system described herein.

Disclosed herein are methods of producing one or more human immune cells. In some aspects, the methods can comprise administering estrogen or estradiol to the immunocompetent genetically modified mice described herein or the transgenic mice described herein between day 7 and 21. In some aspects, the methods can comprise administering estrogen or estradiol to the immunocompetent genetically modified mice described herein or the transgenic mice described herein at different time points. In some aspects, the different time points can include two or more time points between day 7 and 21. As described herein, the administration estrogen or estradiol can be provided to at one or more different time points to boost maturation of immune elements or immune system and/or immune response.

Disclosed herein are methods of producing one or more human antibodies. In some aspects, the methods can comprise introducing at least one candidate antigen into the transgenic mice described herein; and recovering B cells and antibody-producing cells from the transgenic mice described herein. In some aspects, the methods can further comprise rendering the B cells and antibody-producing cells into a single cell suspension; and generating an immortalized cell line from the single cell suspension. In some aspects, the immortalized cell line can be a hybridoma cell line.

Disclosed herein are methods of assessing a human immune response in a mouse. In some aspects, the methods can comprise: a) exposing a transgenic mice described herein to a candidate antigen. In some aspects, the candidate antigen can be associated with a disease or condition. In some aspects, the candidate antigen can be any antigen. In some aspects, the candidate antigen can be known. In some aspects, the candidate antigen can be unknown. In some aspects, the method can comprise taking a biological sample from the transgenic mouse exposed to the candidate antigen. In some aspects, the method can comprise analyzing the biological sample using an assay to measure the immune response in the mouse to the candidate antigen. Examples of assays that can be used to measure the immune response in the mouse to the candidate antigen can include but is not limited to one or more of the following: T cell-dependent and T cell independent antibody response (ELISA) with various model antigens, including for immune stimulation assessment; extended histopathological examination: lymphoid tissues and organs; immunophenotyping in blood or organs (flow cytometry); neutrophil/macrophage (flow cytometry): oxidative burst activity, phagocytosis, and migration; cytokine/chemokine (ELISA, flow cytometry): profiling and release; cell proliferation (beta counter, flow cytometry); natural killer (NK) cell activity (gamma counter, flow cytometry); basophil (flow cytometry); T-cell cytotoxicity; ADCC/CDC (Antibody-Dependent Cell-mediated Cytotoxicity and Complement Dependent Cytotoxicity) assays; ELISpot and FluoroSpot; extracellular markers of cell activation, indicators of cell injury and death, and receptor occupancy and test article binding (flow cytometry); immunohistochemistry and tissue cross-reactivity; anti-dsDNA and antinuclear antibodies; and screening for autoantibodies and autoimmune reactions.

### EXAMPLES

### Example 1

### Generation of Humanized NSGW41 Mice Supporting T-Dependent and T-Independent Class-Switched and Hypermutated Antibody Responses, as Potentiated by Estrogen

Abstract. Animal models are tools in biomedical research with mice being one of the most widely used surrogates of human biology. Although mouse models recapitulate many characteristics of human biological systems, certain aspects are inconsistent with human biology, particularly in the immune system. These divergences include differential TLR expression, species-specific pathogenesis, immune responses, and drug interactions. Traditionally, human studies have been limited to ex vivo and in vitro analyses or costly clinical trials. Thus, underscoring the need for an in vivo model that faithfully recapitulates the human immune system. Humanized mice have made significant progress towards filling this void, allowing for the study of human-specific infections, autoimmune disorders, cancer, allergy and immunity. Through intracardiac transplantation of human umbilical cord-derived hematopoietic stem cells into immunodeficient NSGW41 mice, engraftment levels up to 95% were achieved with robust lymph node development and increased splenic size. Additionally, the kinetics of the human leukocyte development and differentiation within humanized mice was identified. Following immune system development, these humanized mice display serum antibody titers comparable to wild type mice. B cells fully mature within these humanized mice and are capable of robust class switch DNA recombination, memory B cell generation, plasma cell differentiation and specific T-dependent and T-independent antibody responses. Thus, as described herein a robust functional human immune system in vivo platform was generated for translational humoral immunity research and vaccine development.

Introduction. Small animals have been widely used as model systems of human biology due to their size, short reproductive cycles, genomic and physiological similarities to humans and ease of genetic manipulation. While a vast amount of basic biology has been obtained from mouse studies, there are limitations to mouse models when investigating human biology. Several components of mouse biological systems are incongruent with those of humans, particularly within the immune system¹. With more than 1600 genes involved in the innate and adaptive immune system, it is no surprise that there are species-specific differences. Traditionally, human studies have been limited to ex vivo and in vitro analyses or costly and restricted clinical trials. Therefore, outlining the need for an in vivo model that faithfully recapitulates human immunobiology. Humanized mice have made significant progress towards filling this void, allowing for the study of human-specific infections, autoimmune disorders, cancer, allergy and immunity.

Humanized mouse models of the human immune system (H-Mice) are developed by transplanting human hematopoietic stem cells (huHSCs) into immunodeficient recipient mice. Currently available immunodeficient mouse strains enable robust xenotransplantation due to a lack of T, B, and NK cells as well as defective macrophages and dendritic cells which is a result of null genetic mutations in the Il2rg and either Rag or Prkdc genes (NSG mice). Traditionally, sublethal irradiation was required prior to chimerism however, mutation of Kit receptor required for normal hematopoiesis, supports multilineage engraftment without prior irradiation (NSGW41 mice). While multiple sources of human huHSCs have been utilized for the generation of H-Mice, umbilical cord blood offers a highly enriched source of huHSCs leading to a more functional reconstituted immune system than adult bone marrow or fetal liver derived huHSCs, with less ethical concerns.

For H-Mice to fill the current void in human immunobiological research, a functional human immune system with mature cell lineages required to provoke both cellular and humoral immune responses must be established. While B cell responses in vivo have been generated with antigen-specific IgM antibody production, class-switch DNA recombined, affinity matured and antigen-specific antibody responses have yet to be established. Additionally, current H-Mice models have shown inadequate production of memory B cells and plasmablasts.

Disclosed herein are transgenic mice (e.g., H-Mice) established as a functional in vivo platform of the human immune system in both healthy and disease conditions that can be applied to the epigenetic regulation of antibody and autoantibody production. The data disclosed herein demonstrates full reconstitution of NSG and NSGW41 immunodeficient mice with mature human lymphocytes and have identified the kinetic development and differentiation of human leukocytes and the organogenesis of the lymphatic system. The human immune system in the H-Mice respond to both in vitro and in vivo activation to produce class-switched, somatically hypermutated antigen specific antibodies. Additionally, it was demonstrated that the H-Mice mount a fully mature antibody response complete with the production of memory B cells and plasma cells.

Results. To establish a reconstituted human immune (HIS) platform, human umbilical cord blood samples were obtained as a source of human hematopoietic stem cells (HSCs). Umbilical cord-derived HSCs (≥98% purity) were injected intracardially into immunodeficient recipient mouse pups between 24-48 hours of age. Compared to the 51% peak reconstitution in NSG, NSGW41 H-Mice mice maintain a mean peripheral blood reconstitution of 78%, with some reaching 95% of total CD45 expressing cells (FIGS. 1A,C). Reconstitution shown by representative FACS plots of huNSG and NSGW41 H-Mice at 10, 20 and 30 weeks of age (FIG. 1B). At 20 weeks of age, NSGW41 H-mice reach an average of 3.9×10⁶ human CD45⁺ cells per ml, with some mice reaching 8.34×10⁶ (FIG. 1D). NSGW41 H-Mice exhibit expanded B cell and myeloid lineage compartments of human leukocytes: 2.25-2.75*10⁶ B cells per ml, 1.5-2*10⁶ T cells per ml, 2-2.5*10⁵ dendritic cells per ml, 1-2*10⁵ monocyte/macrophage cells per ml, 1-2*10⁵ NK cells per ml (FIGS. 1E,F). Concomitantly, NSGW41 H-Mice maintain 13-16% human CD34⁺ stem cells in the bone marrow (FIG. 1G). Suggesting increased reconstitution in NSGW41 H-Mice could be due to a sustained stem cell reservoir.

Construction of humanized mice. The development of an in vivo platform with a fully functioning human immune system is important to the translational research of human immunology. To establish a humanized mouse platform, human umbilical cord blood samples were obtained as an ethical source of human hematopoietic stem cells (huHSCs). Following healthy full-term births, CD34⁺ huHSCs were isolated within 30 minutes post-partum, achieving optimal viability and stemness, which is an important component of this platform. Umbilical cord-derived huHSCs (≥98% purity) were intracardially transplanted into either irradiated NSG or non-irradiated NSGW41 pups at 48 hours of age.

The NSGW41 H-Mice platform supports significantly greater reconstitution and long-term persistence of the human immune system than their irradiated NSG counterparts, as defined by peripheral blood analysis over time. Compared to the 51% peak reconstitution in NSG mice, NSGW41 mice maintain a mean reconstitution of 78%, with some reaching 95%. Additionally, NSG mice reconstitution sharply declines after peaking at 20 weeks, whereas NSGW41 reconstitution persists through 40 weeks of age. NSGW41 H-Mice support a greater proportion of huHSC-derived cells, and retain drastically greater human leukocyte counts (5.6 million/ml at weeks of age), as determined by CBC analysis.

While NSGW41 mice show greater human reconstitution, it was desired to evaluate the differentiation-capacity of these cells and determine the composition of the human immune system. Thus, the relative percentages of B cells and T cells were characterized within the peripheral blood, spleen and lymph nodes. The characterization was furthered by calculating the cells per ml within the peripheral blood for total human CD45 expressing cells, B cells, T cells dendritic cells, monocytes/macrophages and NK cells. NSGW41 mice maintain a drastically greater B cell compartment than their NSG counterparts, which is a marked improvement over previous models which have an established B lymphocyte deficiency. Furthermore, at 6- and 25-weeks post-transplant, NSGW41 mice support considerably greater CD34⁺ engraftment within the bone marrow as compared to NSG mice (2.4 and 9.7-fold increase in CD34⁺ cells). Suggesting increased reconstitution of NSGW41 mice could be due to a sustained reservoir for human immune system reconstitution.

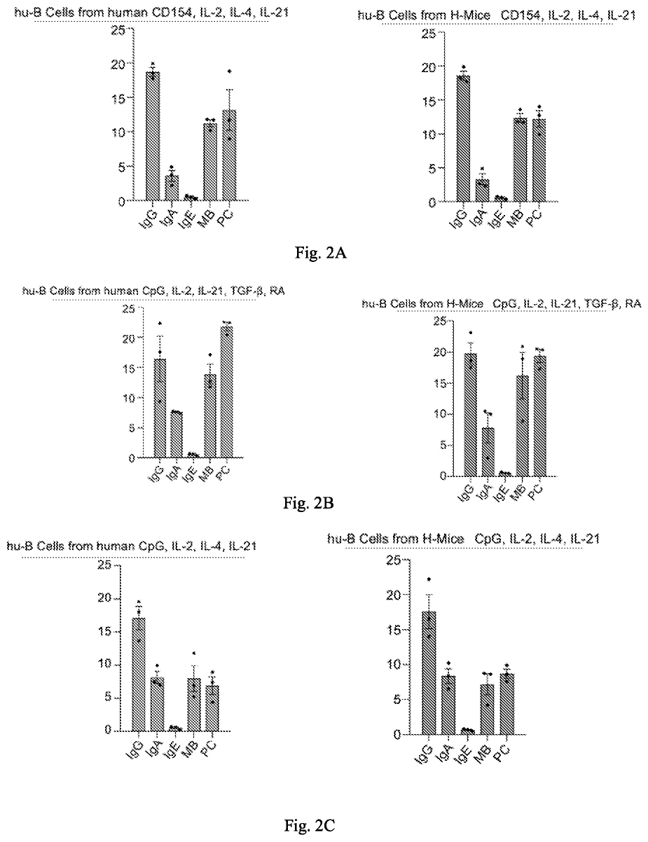

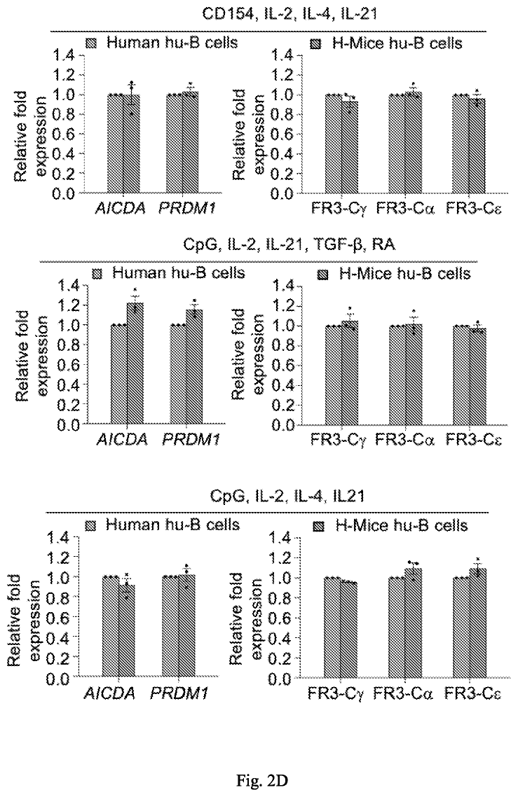

NSGW41 H-Mice B cells undergo CSR and plasma cell differentiation as efficiently as adult human B cells. To investigate the functionality of human B cells developed in NSGW41 H-Mice; B cells were isolated from NSGW41 H-Mice and human doners and compared to their ability to undergo CSR and plasma cell differentiation in response to in vitro stimulation with CD154 or CpG. When stimulated with CD154, IL-2, IL-4, and IL21, NSGW41 H-Mice class-switch and differentiate into plasma cells as effectively as adult human B cells (FIG. 2A). B cells isolated from NSGW41 H-Mice and adult human donors were also stimulated with TLR9 ligand, CpG, with IL42 IL-21 TFG-β and retinoic acid (RA) (FIG. 2B) and with CpG, with IL-2, IL-4, and IL-21 (FIG. 2C). Across the three activation conditions, NSGW41 H-Mice B cells undergo CSR and plasma cell differentiation as efficiently as those isolated from human donors. Additionally, NSGW41 H-Mice B cells had equivalent expression of AICDA and PRDM1 transcripts as well as post-recombination FR3-Cγ, FR3-Cα and FR3-Cε transcripts when compared to adult human B cells (FIG. 2D). Therefore, CD40 activation by CD154 and TLR engagement induces CSR and plasma cell differentiation at equivalent levels in NSGW41 H-Mice B cells as in those isolated from healthy adult human donors. Thus, suggesting, B cells developed within NSGW41 H-Mice fully mature to undergo CD154- and TLR-induced CSR and plasma cell differentiation.

NSGW41 mice have naïve B cell repertoire comparable to adult humans. B cell maturation and productive Ig gene rearrangement to generate a highly diverse, polyclonal immunoglobulin repertoire is important for B cell recognition of a diverse range of antigens. To investigate B cell receptor repertoire diversity in NSGW41 H-mice compared to humans, the expression of Ig V_{H}, D, J_{H} and Vκ, Jκ, as well as Vλ, Jλ genes were analyzed in CD19⁺ B cells isolated from healthy adult donors and NSGW41 H-Mice. The relative usage of variable, diversity and joining gene segments in NSGW41 H-mice was comparable to those of healthy human adults. Taken together, this data suggests human B cells developed within NSGW41 H-Mice generate B cell repertoire diversity equivalent to healthy human adults.

NSGW41 H-Mice mice mount a mature antibody response to both T-dependent and T-independent antigens. While in vitro activation addresses the development of human B cells within NSGW41 H-Mice, the ability of NSGW41 H-Mice to mount a mature antibody response was investigated. While previous humanized mouse models have exhibited low affinity IgM antibody titers, they lack appreciable (CSR) and affinity maturation conferring high-affinity antibody production, thus limiting their applications.

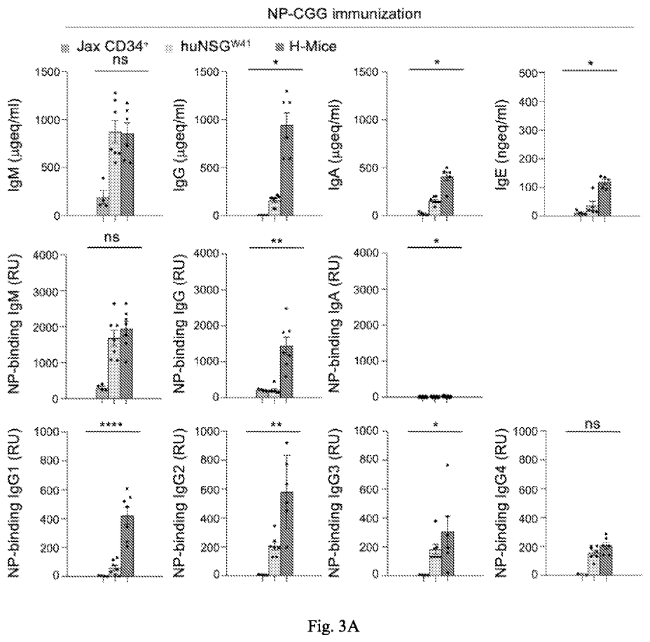

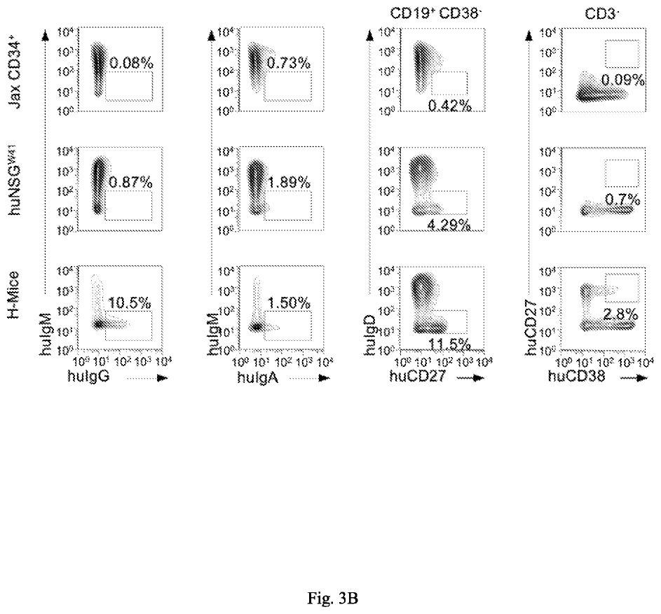

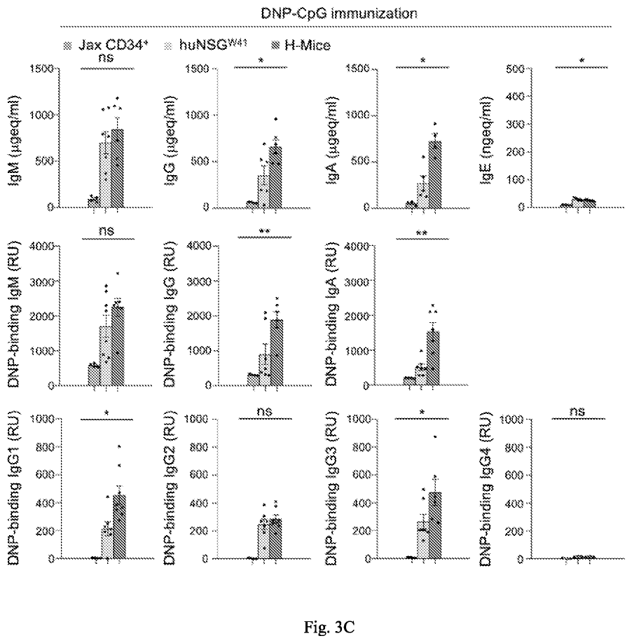

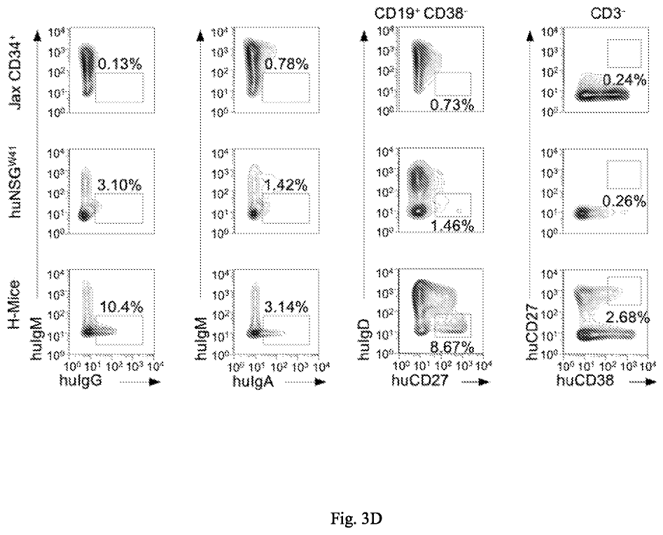

To address the antibody response within NSGW41 H-Mice, 16 NSGW41 H-Mice were segregated into two groups of 8 mice each. One group was fed water containing estrogen (NSGW41 H-Mice) and the other plain water (huNSG^{W41}), each of them were then i.p. injected with NP₁₆-CGG and Alum. The NSGW41 H-Mice were compared to Jackson Labs CD34⁺ humanized mice (Jax CD34⁺). Following immunization, the huNSG^{w41} and NSGW41 H-Micehad comparable IgM serum titers falling within the normal serum reference intervals of healthy adults. However, the Jax CD34⁺ had dramatically lower titers, correlating with reduced human reconstitution. Class-switched IgG, IgG1-4, IgA and IgE production was drastically increased in NSGW41 H-Mice receiving E2-water. Furthermore, NSGW41 H-Mice given E2-water had significantly greater NP₇-binding IgM, and IgG antibody titers (FIG. 3A). The E2-mediated increase in switched NP₇-binding antibodies reflected the increased number of spleen IgG and IgA class-switched effector, IgD⁻CD27⁺ memory B cells, and bone marrow CD27⁺CD38⁺ plasmablasts/plasma cells. (FIG. 3B).

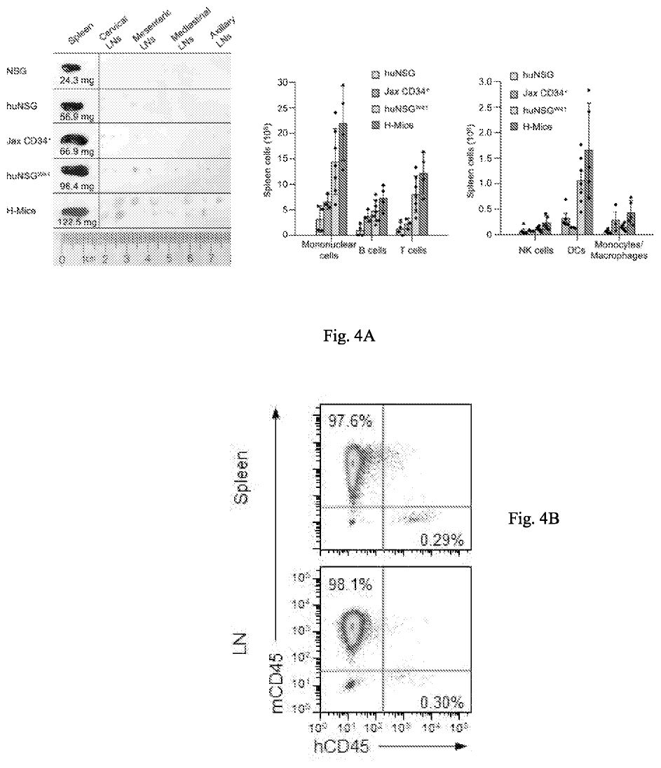

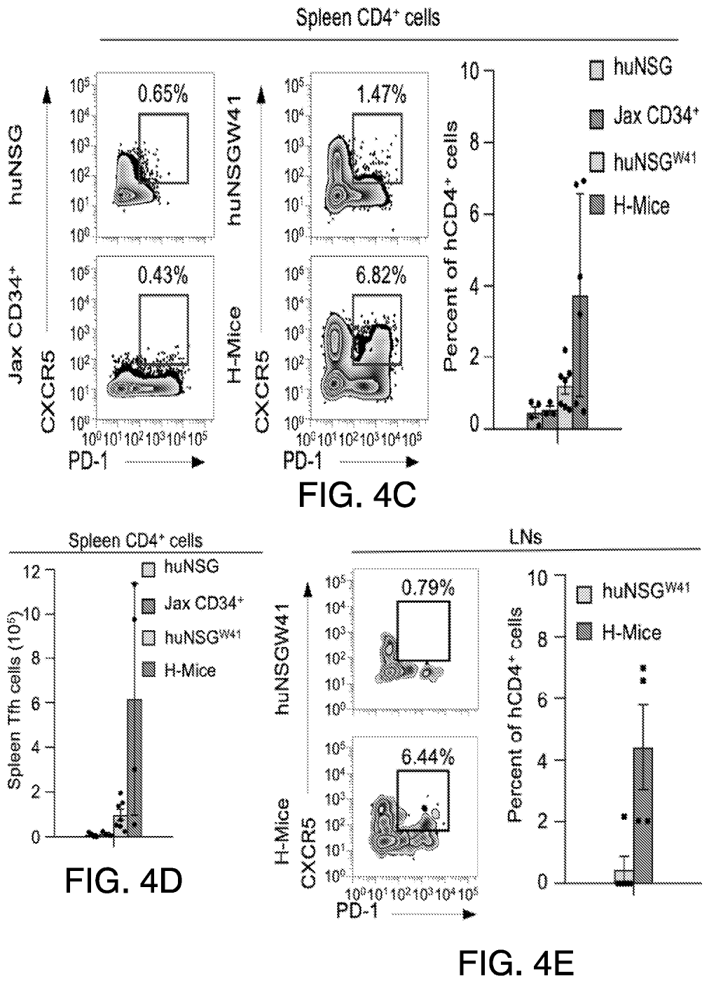

While NP₁₆-CGG evaluates the T cell-dependent response, the NSGW41 H-Mice immune system's ability to generate a T cell-independent antibody response was investigated. To do so, 16 mice NSGW41 H-Mice were segregated amongst the E2 and plain water groups and injected with Haptenated TLR9 ligand, DNP-CpG IP. As observed in the NP-CGG injected NSGW41 H-Mice, total IgM production was comparable between huNSG^{w41} and NSGW41 H-Mice, while E2 dynamically potentiated the class-switched IgG and IgA antibody production. Additionally, DNP₄-binding IgM, IgG, and IgA titers were significantly increased in E2 administered group (FIG. 4c). The increased class-switched and antigen binding antibody production was concomitant with increased numbers of IgG⁺ and IgA⁺ B cells, as well as splenic IgD⁻CD27⁺ memory B cells, and bone marrow CD27⁺CD38⁺ plasmablasts/plasma cells (FIG. 3D). Thus, NSGW41 H-Mice receiving E2 treatment can mount a mature antibody response to T-dependent and T-independent antigens complete with class-switched, high affinity antibody production and generation of both plasma and memory B cells.

NSGW41 H-Mice have superior lymphoid organogenesis and spleen reconstitution.

NSGW41 H-Mice show dramatically greater total mononuclear cells per spleen (22.0×10⁶ vs 6.58×10⁶; H-Mice® and Jax CD34⁺, respectively) as well as expanded B, T, Dendritic cell, and monocyte/macrophage compartments (FIG. 4A). These findings are concomitant with 98% human cells within the spleen and lymph nodes (FIG. 4B). Additionally, NSGW41 H-Mice demonstrate greater proportions and numbers of T follicular helper (T_{FH}) cells within the spleen and lymph nodes (FIGS. 4C,D,E).

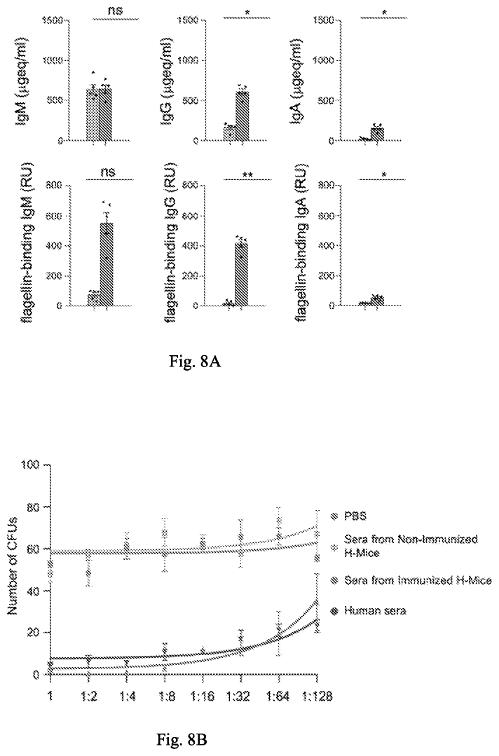

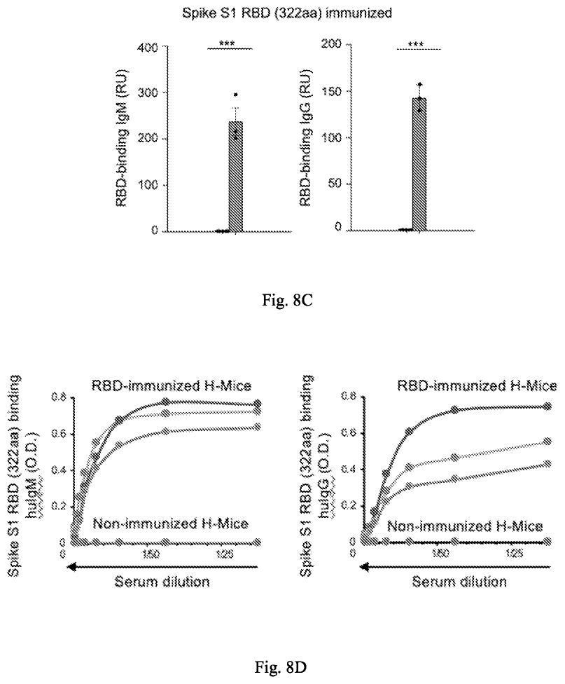

Given these findings, the next step was to move from model antigens to immunization with disease relevant bacterial and viral components. To do so, it was investigated whether NSGW41 H-Mice could mount a mature, neutralizing antibody response following immunization with purified Salmonella typhimurium flagellin. NSGW41 H-Mice were injected intraperitoneally with 50 μg of flagellin in alum and measured flagellin-specific antibody titers by ELISA. As shown in FIG. 8A, NSGW41 H-Mice responded to flagellin to generate increased IgG and IgA total serum antibody titers. Notably, NSGW41 H-Mice generate potent IgM and IgG flagellin-binding antibodies, indicative of a class-switched antibody response induced by flagellin, which has not been reported previously. Next, the immune sera, consisting of flagellin-specific antibodies to assess its ability to mediate microbial killing in vitro by performing a serum bactericidal activity assay, was examined. The results show that immune sera from flagellin-immunized NSGW41 H-Mice killed a 200 CFU dose of live Salmonella typhimurium in vitro as efficiently as human donor sera with positive anti-flagellin titers (FIG. 8B). A dose-dependent increase in the number of bacterial colonies remaining wasn't observed until a 1:64 dilution of both NSGW41 H-Mice human sera. Thus, the data show that following immunization, NSGW41 H-Mice generate flagellin-specific antibodies, induced by injection of purified flagellin, are functionally capable of mediating bacterial killing in vitro.

Then NSGW41 H-Mice antibody response to viral antigens was investigated. To do so, 20-week-old, fully reconstituted NSGW41 H-Mice were immunized with 50 μg of recombinant SARS-CoV2 S1 spike protein receptor binding domain (RBD) in alum. NSGW41 H-Mice were boosted with a second 50 μg injection of RBD 7 days later in PBS. Our data demonstrate NSGW41 H-Mice mount a class-switched, high-affinity antibody response to the RBD domain of the SARS-CoV2 S1 spike protein (FIGS. 8C and 8D).

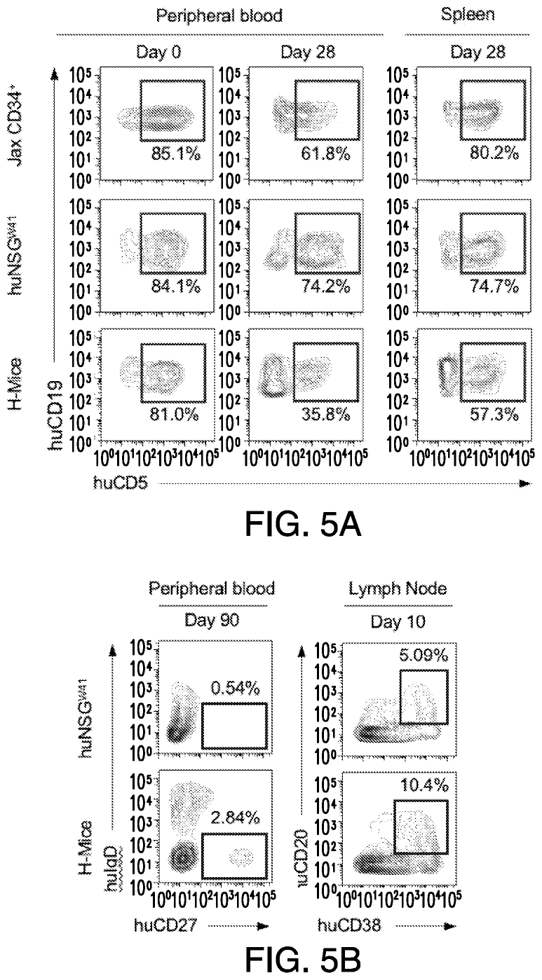

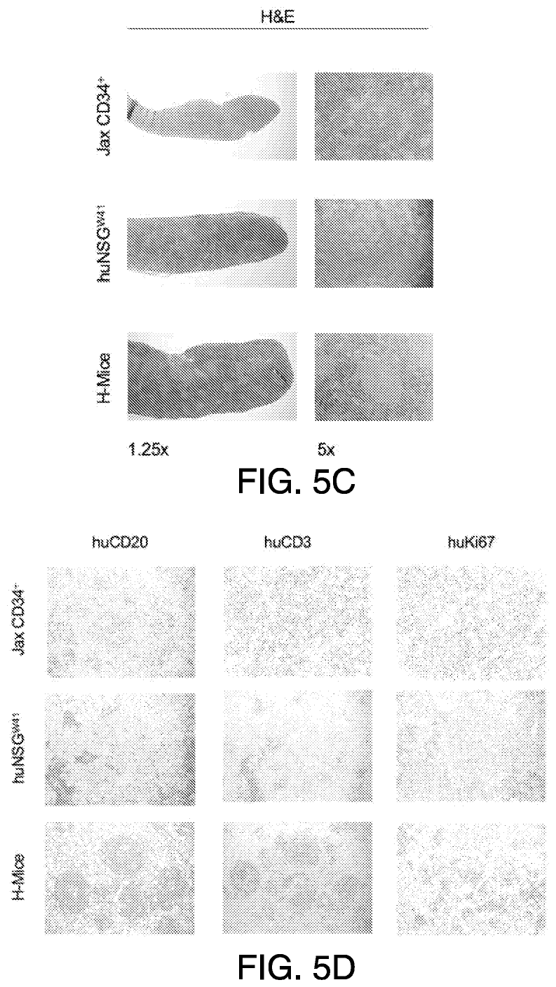

E2 promotes myeloid lineage differentiation and B2 proliferation. Since estrogen administration had such a dynamic impact on the maturation of antibody response within NSGW41 H-Mice, the cellular composition and lymphoid architecture. B1/B2 cell ratios were compared across HIS platforms following immunization with NP-CGG. NSGW41 H-Mice exhibited a drastic inversion of the B1/B2 cell ratios within the peripheral blood (81.0% to 35.8% B2 cells) while other HIS platforms showed little change. NSGW41 H-Mice also showed significantly lower B2 ratios in the spleen, suggesting preferential differentiation of B1 cells in response to immunization (FIG. 5A). NSGW41 H-Mice also showed circulating memory B cells (CD19⁺CD38⁻IgD⁻CD27⁺) through 90 days post-immunization and a two-fold increase in germinal center B cells 10 days-post immunization (FIG. 5B). To investigate the formation of lymphoid follicles and germinal center structures, Jax CD34⁺, huNSG, and NSGW41 H-Mice spleens were extracted 10 days post-immunization. H&E staining shows clear follicular formations within NSGW41 H-Mice, while neither Jax CD34⁺ or huNSG mice spleens exhibit any defined structures (FIG. 5C). These findings are supported by IHC staining of huCD20, huCD3 and huKI67 showing germinal center structures in NSGW41 H-Mice (FIG. 5D).

Estradiol potentiates AICDA and PRDM1 expression and somatic point mutation frequency. A defining feature of a mature antibody response is somatically mutated IgV genes, indicating that they have undergone multiple iterations of antigen-driven selection within a GC reaction.

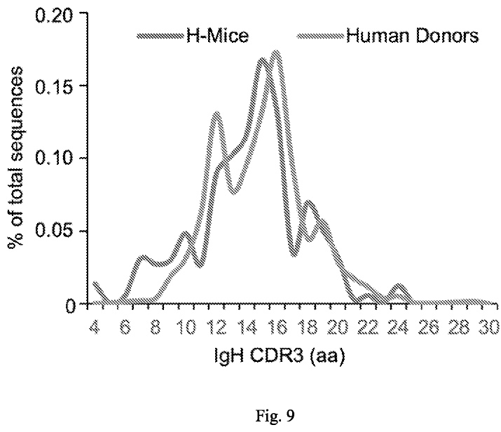

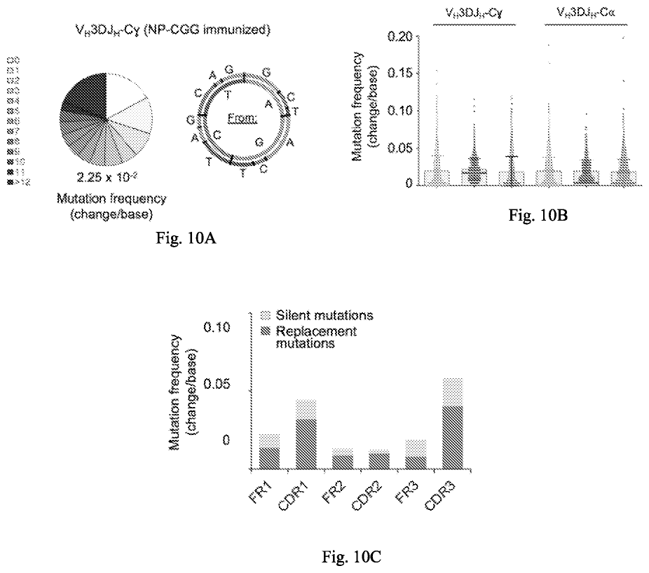

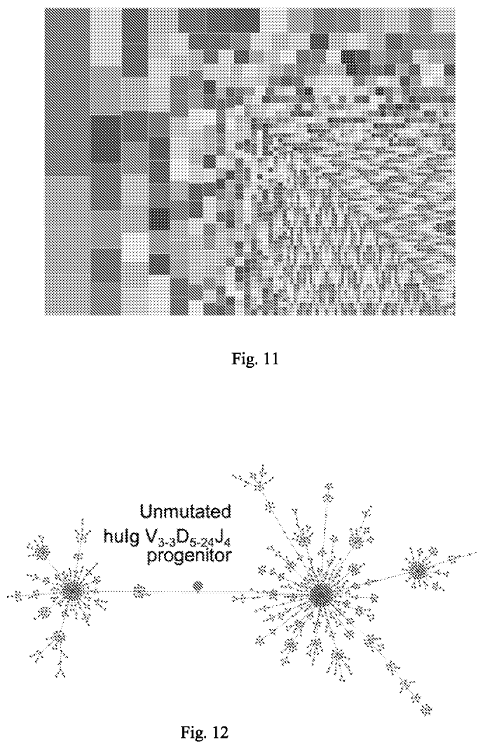

Somatic IgH V_{H}DJ_{H} rearrangement determines the sequence and length of the complementary determining region 3 (CDR3), which is important for BCR-antigen contact. To address IgH CDR3 length and nature as well as V_{H} mutational load, recombined V_{H}DJ_{H}-C_{H} transcripts were amplified using forward (degenerate) primers for V_{H}1 and V_{H}3 gene leader sequences in conjunction with reverse Cg or Ca isotype-specific primer and analyzed by IMGT/HighV-QUEST. The distribution of NSGW41 H-Mice CDR3 lengths largely overlapped with those from healthy adult donors (FIG. 9). A salient feature of a high-affinity antibody response is the load of somatic-point mutations in expressed Ig V(D)J genes, a result of precursor B cells undergoing antigen-driven SHM and selection. Transcripts from class-switched, IgG⁺ NSGW41 H-Mice B cells exhibit a robust frequency of somatic point-mutations (0.0225 change/base) with a stochastic distribution of nucleotide replacements, suggesting proper functioning of DNA-repair machinery following initiation of deamination by AID (FIG. 10A). Both Cγ and Cα transcripts, from three separate NSGW41 H-Mice, demonstrate a broad distribution of mutations per transcript (FIG. 10B). Notably, the CDR regions carried a greater mutational burden then framework regions, with a larger proportion of replacement mutations, further suggesting antigen-driven selection is occurring (FIG. 10C). To evaluate clonal expansion, V_{H}DJ_{H}-Cγ transcripts were placed into groups based on identical CDR3 sequence and identical V_{H}DJ_{H} gene segment usage and quantified with the size of the rectangle representing the number of unique transcripts. Following immunization, NSGW41 H-Mice B cells undergo clear clonal expansion of select progenitors (FIG. 11). Furthermore, phylogenic mapping demonstrates complex clonal expansion from unmutated progenitors (FIG. 12). These data suggest NSGW41 H-Mice undergo a mature human antibody response, complete with SHM, antigen-driven selection, and clonal expansion.

Methods. Mice. NSG (NOD.Cg-Prkdc^{scid} Il2rg^{tm1Wjl}/SzJ, 005557)¹⁷ and NSGW41 (NOD.Cg-Kit^{W-41J} Prkdc^{scid} Il2rg^{tm1Wjl}/WaskJ)¹⁸ mice were purchased from the Jackson Laboratory (Bar Harbor, Me.), and were housed and bred in our pathogen-free vivarium.

Human Donor Cells. Human umbilical CB samples were obtained from healthy full-term births in the Department of obstetrics and gynecology of the University Hospital, University of Texas Health Science Center at San Antonio.

Engraftment. Human CD34⁺ progenitor cells were isolated from human CB by positive selection using the EasySep Human Cord Blood CD34 Positive Selection Cell Isolation Kit (STEMCELL Technologies) following the manufacturer's instructions. For human engraftment, 1×10⁵ CD34⁺ cells were injected intracardially in 50 μL of PBS/2% fetal calf serum into 48-hour-old sublethally irradiated NSG (100 cGy) or non-irradiated NSGW41 mice.

Blood Analysis. The mice were bled via venous puncture no earlier 6 weeks post-engraftment to check for reconstitution of the human immune system. For complete blood counts, blood of mice was collected in EDTA-containing microtubes and immediately analyzed on a Sysmex XT2000iVblood analyzer.

Immunizations. NSGW41 H-Mice were injected intraperitoneally with 4-hydroxy-3-nitrophenylacetlyl (NP) conjugated to CGG (NP-CGG) (average: 16 NP molecule conjugated with one CGG molecule; Biosearch; 100 μg) in PBS (100 μl) with Alum (100 μl), or 2,4-Dinitrophenyl conjugated to CpG (DNP-CpG) (1 DNP molecule conjugated with one CpG molecule; Eurofins Genomics; 25 μg) in PBS (100 μl) without adjuvant. Sera were collected before injection and at specified time points after injection.

E2 Administration. NSGW41 H-Mice were given drinking water ad libitum containing 1 μM estradiol. Drinking water at or above this concentration was well accepted by the mice showed no adverse effects.

Histology. Spleen and intestinal tissues were harvested and then sectioned and H&E stained at UTHSCSA histology core facilities. Images were captured using a Zeiss Imager-V.1. Tissue histology sections were examined and histological grading was performed by a UTHSCSA pathologist. Sections were scored as follows: 0—Normal; 1—Normal with focal epithelial loss; 2—Normal with mild surface epithelial loss; 3—Partial necrosis and diffuse epithelial loss; 4—Total necrosis.

A total of 100 μg of chicken OVA (Sigma-Aldrich) in 100 μL of phosphate-buffered saline (PBS) was mixed with an equal volume of Complete Freund's Adjuvant (Difco); and 14-week-old humanized mice were immunized by IP injection. Two weeks later, mice were boosted with 100 μg of OVA in 100 μL of PBS mixed with an equal volume of Incomplete Freund's Adjuvant (Difco). Seven to 10 days later, mice were bled to analyze the levels of antigen-specific immunoglobulins and boosted again with OVA+Incomplete Freund's Adjuvant. Seven to 10 days after the last booster, the mice were euthanized and tissue samples were collected.

Detection of antibodies. Titers of IgM, IgD, IgG and IgA from in vitro culture supernatants of stimulated human and H-Mice B cells or titers of circulating and/or NP-binding/DNP-binding IgM, IgG, and IgA were measured using specific ELISAs. IgE were detected by sandwich ELISAs, using plates coated with anti-human IgE mAb. Serial two-fold diluted serum samples in PBS-(0.05%) Tween 20 (PBS-Tween 20) were added to the plates and incubated for 2 h at 23° C. After washing with PBS-Tween 20, biotin-detection antibody was added. After a final washing, IgE antibodies were detected using streptavidin-horseradish peroxidase. Antigen specific antibody titers are expressed as relative units (RU), defined as the dilution factor needed to reach 50% saturation binding, as calculated using GraphPad Prism® software (GraphPad).

Detection of antibody forming cells (AFCs). Spleen, bone marrow, mesenteric lymph node and Payer's patch cells were isolated from desired mice and analyzed for AFCs by ELISPOT. MultiScreen® ELISPOT plates (MAIPS4510, Millipore) were activated with ethanol (35%), washed four times with PBS and coated with unlabeled rabbit polyclonal antibodies against mouse IgM, IgG1, or IgA in PBS overnight at 4° C. The plates were then washed six times with PBS, blocked with BSA (0.5%) in RPMI/HEPES+L-glutamine for 1 h at room temperature. Single cell suspensions from lamina propria, bone marrow and spleen cells of OVA-immunized mice were cultured in the plates at 37° C. for 16 h in FCS-RPMI at 250,000, 125,000 and 75,000 cells/well. The cultures were then removed, the plates were washed 6 times, incubated with biotin-anti-IgM, IgG1, or IgA for 2 h on a shaker at room temperature, washed, incubated with horseradish peroxidase (HRP)-streptavidin (Santa Cruz Biotech) for 1 h on a shaker at room temperature, washed again and developed using the Vectastain AEC peroxidase substrate kit following manufacturer's protocol (SK-4200, Vector Laboratories). Plates were imaged and quantified using a CTL-ImmunoSpot® Analyzer and software.

Flow cytometry. Total B cells and PBMCs were subject to flow cytometry for pre-and-post isolation phenotypic analysis using the following surface markers and fluorophores: PEcy7-anti-human-CD19 (clone H1B19; Biolegend), PE-anti-human-CD27 (clone M-T271; Biolegend), BV421-anti-human-IgD (clone HB-7; Biolegend), FITC-anti-human-IgG (clone G18-145; BD Pharmingen), APC-anti-human-IgA (clone IS11-8E10; Miltenyi Biotec) and APC-Cy7-anti-IgM (clone MHM-88, Biolegend) and run on a BD LSRII (BD Biosciences) with the FACSDiva software (BD Biosciences). Data were analyzed using FlowJo software (FlowJo LLC).

Approximately 5×10⁷ total B cells enriched from PBMCs were used for cell sorting. 75% of the total B cells were stained with FITC-anti-human-IgG (clone G18-145; BD Pharmingen), APC-anti-human-IgA (clone IS11-8E10; Miltenyi Biotec), and PE-anti-human-CD27 (clone M-T271; Biolegend) for isolation of class-switched memory B cells. CD27⁺IgG⁺ and CD27⁺IgA⁺ populations were sorted into 1.2 mL Eppendorf tubes containing 0.5 mL of HBSS (Hank's balanced salt solution) buffer containing 0.1% Bovine serum albumin (BSA). 25% of total B cells were stained with anti-IgD BV421 (clone HB-7; Biolegend) and anti-CD27 PE (clone M-T271; Biolegend); CD27⁻IgD⁺ and CD27⁺IgD⁺ populations were sorted in 0.5 mL of HBSS+0.1% BSA buffer.

For intracellular staining, B cells were fixed in 150 μl of formaldehyde (3.6%) for 10 min at 25° C. In the case of IgE intracellular staining, cells were trypsinized before formaldehyde fixation. Fixed cells were then permeabilized in cold methanol (90%) for 30 mins on ice before staining with VF-anti-CD19 mAb (75-0193-0100, Tonbo), FITC-anti-AID Ab (bs-7855R-FITC, Bioss), APC-anti-Blimp-1 mAb (5E7, BioLegend), PE-Cy7-anti-CD138 mAb, APC-anti-IgG1 mAb (406610, BioLegend), FITC-anti-IgG2a mAbs (553390, BD Biosciences) and/or PE-anti-IgE mAb (23G3, eBioscience). FACS analysis was performed on single cell suspensions. In all flow cytometry experiments, cells were appropriately gated on forward and side scattering to exclude dead cells and debris. Cell analyses were performed using a LSR-II flow cytometer (BD Biosciences), and data were analyzed using FlowJo software (TreeStar). The experiments were performed in triplicates.

Human and NSGW41 H-Mice B cells, CSR and plasma cell differentiation. NSGW41 H-Mice IgD⁺ naïve B cells were isolated from 20 weeks-old HuNSGW41 mice. B cells were resuspended in FCS-RPMI containing 50 mM β-mercaptoethanol and 1× antibiotic-anti-mycotic mixture (15240-062, Invitrogen) at 37° C. in 48-well plates and stimulated with CpG (2 ng/ml) or CD154 (1 U/ml, obtained from membrane fragments of CD154 encoding recombinant baculovirus-infected Sf21 insect cells) plus IL-4 (5 ng/ml, R&D Systems) for CSR to IgG, IgA, IgE, and plasma cell differentiation and cells were collected at various times.

Human IgD⁺ naïve B cells (˜99% pure) were purified by negative selection from healthy donor PBMCs using the EasySep Human Naïve B Cell Enrichment Kit (19254, STEMCELL Technologies), following the manufacturer's instructions. Naïve B cells were then cultured in FCS-RPMI and stimulated with trimeric CD154 (10 U/ml), IL-4 (20 ng/ml, R&D Systems) and IL-21 (50 ng/ml, R&D Systems) or CD154 (10 U/ml), IL-21 (50 ng/ml) and TGF-β (0.5 ng/ml) for 120 hrs. B cells were then stained with 7-aminoactinomycin D (7-AAD), FITC-anti-IgM mAb (314506, Biolegend), PE-anti-CD19 mAb (302208, BioLegend) and allophycocyanin-anti-IgG mAb (562025, BD Biosciences) or 7-AAD, FITC-anti-IgA mAb (F5259, Sigma-Aldrich), and biotin-F(ab′)₂ anti-IgM (2022-08, Southern Biotech), followed by allophycocyanin-streptavidin, and analyzed by flow cytometry.

Quantitative RT-PCR of mRNA, miRNA, germline, post-recombination and mature transcripts. For quantification of mRNA, pri-miRNA, germline I_{H}-C_{H}, post-recombination Iμ-C_{H} and mature V_{H}DJ_{H}-C_{H} transcripts, RNA was extracted from 0.2-5.0×10⁶ cells using either Trizol® Reagent (Invitrogen) or RNeasy Plus Mini Kit (Qiagen). Residual DNA was removed from the extracted RNA with gDNA eliminator columns (Qiagen). cDNA was synthesized from total RNA with the SuperScript™ III First-Strand Synthesis System (Invitrogen) using oligo-dT primer. Transcript expression was measured by qRT-PCR with the appropriate primers using a Bio-Rad MyiQ™ Real-Time PCR Detection System (Bio-Rad Laboratories) to measure SYBR Green (IQ™ SYBR® Green Supermix, Bio-Rad Laboratories) incorporation with the following protocol: 95° C. for 15. sec, 40 cycles of 94° C. for 10 sec, 60° C. for 30 sec, 72° C. for 30 sec. Data acquisition was performed during 72° C. extension step. Melting curve analysis was performed from 72-95° C. For quantification of mature miRNA transcripts, RNA was extracted from 0.2-5.0×10⁶ cells using miRNeasy® Mini Kit (Qiagen) and then reverse-transcribed with miScript II RT Kit (Qiagen) using the miScript HiSpec buffer. A Bio-Rad MyiQ™ Real-Time PCR Detection System was used to measure SYBR Green (miScript SYBR Green PCR Kit, Qiagen) incorporation according to manufacturer's instructions. Mature miRNA forward primers were used at 250 nM in conjunction with the Qiagen miScript Universal Primer and normalized to expression of small nuclear/nucleolar RNAs Rnu6/RNU61/2, Snord61/SNORD61, Snord68/SNORD68, and Snord70/SNORD70. The ΔΔCt method was used for data analysis of qRT-PCR experiments.

RNA sequencing and statistical analysis of B cell and T cell repertoire. RNA was isolated from cells using the Directzol RNA Microprep Kit (Zymogen Research) (based off of cell number), according to manufacturer's instructions. RNA integrity was verified using an Agilent Bioanalyzer 2100 (Agilent). Next generation RNA-Seq for mRNA and non-coding RNA was performed by the Genome Sequencing Facility at the Greehey Children's Cancer Research Institute (UTHSCSA). High-quality RNA was processed using an Illumina TruSeq RNA sample prep kit v2 or TruSeq Small RNA Sample Prep kit following the manufacturer's instructions (Illumina). Clusters were generated using TruSeq Single-Read Cluster Gen. Kit v3-cBot-HS on an Illumina cBot Cluster Generation Station. After quality control procedures, individual mRNA-Seq or small RNA-Seq libraries were then pooled based on their respective 6-bp index portion of the TruSeq adapters and sequenced at 50 bp/sequence using an Illumina HiSeq 3000 sequencer. Resulting reads were checked by assurance (QA) pipeline and initial genome alignment (Alignment). After sequencing, demultiplexing with CASAVA was employed to generate a fastq file for each sample. Initial data processing was performed by the Department of Epidemiology and Biostatistics at the University of Texas Health Science Center at San Antonio. Sequencing reads were aligned against their reference genome (UCSC hg19) using TopHat default settings. Bam files from the alignment were processed using HTSeq-count to obtain counts per gene in all samples. RNA expression levels were determined using GENCODE annotation (GENCODE human v24). mRNA and lncRNA sequencing generated 12-21 million reads per sample, while smRNA sequencing generated 0.6-2.5 million reads per sample. Differential expression analysis was performed using the edgeR package in R post-normalization. mRNA/lncRNA was removed from downstream analysis if they did not break the threshold of at least 1 RPKM mapped reads across all sample libraries. Differentially expressed (DE) mRNA between 2 groups was defined based on a Benjamini Hochberg false discovery rate (FDR)-corrected threshold for statistical significance of padj<0.05. DE of miRNA and lncRNA between 2 groups was defined based on a criterion of p<0.05. Volcano plots depicting loge-fold change and raw or adjusted p values were generated in R. Transcript read counts were transformed to loge RPKM (Reads per Kilobase per Million reads) and used to generate heatmaps as well as PCA plots in Clustvis. Circos plot ideograms were generated using the RCircos package in R.

SHM. To analyze SHM in the sorted fractions, RNA was extracted. cDNA was synthesized from 1-2 μg total RNA with the SuperScript™ III First-Strand Synthesis System (Invitrogen) using oligo-dT primer. Rearranged V_{H}DJ_{H}-C_{H} cDNA was amplified using a V_{H1-6} leader-specific forward primer together with reverse Cγ,α,μ or Cδ isotype-specific primer²⁰ tagged with Illumina clustering adapters and Phusion™ high fidelity DNA polymerase (New England BioLabs). PCR conditions were 98° C. for 10 s, 57° C. for 45 s and 72° C. for 1 min for 30 cycles. The amplified library was tagged with barcodes for sample multiplexing, PCR enriched, and annealed to the required Illumina clustering adapters. High-throughput, 300 bp pair-ended sequencing was performed using the Illumina MiSeq system. Somatic mutations in the V_{H}DJ_{H} region were determined using IMGT/HighV-QUEST.

Statistical analyses. The statistical analyses were performed using Excel (Microsoft) or GraphPad Prism® software. Differences in Ig titers, CSR and RNA transcript expression were analyzed with Student's paired (in vitro) and unpaired (in vivo) t-test assuming two-tailed distributions. Differences in the frequency and spectrum of somatic point-mutations were analyzed with χ² tests. A p value of <0.05 was considered significant.

### Example 2

### Antibody and Autoantibody Response in Humanized Mice and Kinetic Characterization of Human Immune System Development

Humanized mice have made significant progress towards filling this void, allowing for the study of human-specific infections, autoimmune disorders, cancer, allergy and immunity. Through intracardiac transplantation of human umbilical cord-derived hematopoietic stem cells into immunodeficient NSGW41 mice, engraftment levels up to 95% were achieved with robust lymph node development and increased splenic size. Additionally, the kinetics of the human leukocyte development and differentiation within humanized mice was identified. Following immune system development, these humanized mice display serum antibody titers comparable to wild type mice. B cells fully mature within these humanized mice and are capable of robust class switch DNA recombination, memory B cell generation, plasma cell differentiation and specific T-dependent and T-independent antibody responses. Also, these humanized mice can be induced to generate substantial autoantibody production leading to systemic symptoms modeling lupus in a human lymphocyte dependent manner. Thus, a robust functional human immune system in vivo platform was generated for translational humoral immunity research and vaccine development.

Collection of human umbilical cord blood samples. Cord blood samples are collected by collaborating OB/GYN immediately following healthy, full term cesarean section births. Samples are received within 30 minutes post-partum and hematopoietic stem cells are immediately isolated. CD34+ hematopoietic stem cells were isolated. Briefly, hematopoietic stem cells are isolated from human umbilical cord blood by utilizing the StemCell EasySep CD34+ cord blood kit. Cord blood is pre-enriched for HSCs by RosetteSep antibody cocktail which depletes differentiated lymphocytes. MNCs are isolated from pre-enriched cord blood by density gradient centrifugation followed by positive selection of CD34+ cells. Isolation yields 98% HSC purity or greater.

Next, immunodeficient NSG and NSGW41 mice are engrafted at 48 hours of age via intracardiac injection of 1.0×10⁵ hematopoietic stem cells isolated from human umbilical cord blood.

The data show that a human immune system can be developed within NSGW41 H-Mice. Following intracardiac transplant of hematopoietic stem cells, human engraftment was assessed biweekly by flow cytometry analysis of PBMCs expressing either human or mouse CD45. Engraftment levels were depicted as human CD45+ percent of total CD45+ cells. Engraftment levels within spleen and lymph nodes of NSGW41 H-Mice assessed by determining human CD45+ percent of total CD45+ cells.

Human leukocyte differentiation was assessed by flow cytometry analysis of PBMC surface expression of CD45, CD19, CD3, CD11c, CD14 and CD56. The data show human lymphocyte differentiation kinetics.

B cell development. B cells develop within the bone marrow from hematopoietic stem cells and enter the peripheral compartments as naïve B cells. Upon encountering cognate antigen, B cells are activated and undergo somatic hypermutation and class switch DNA recombination and differentiate into either CD27+CD38+ plasma or CD27+CD38− memory B cells.

T-cell dependent and independent CSR-inducing stimuli. CSR entails induction of AID, germline IH-S-CH transcription and active histone modifications in S regions by primary CSR-inducing stimuli together with secondary stimuli (cytokines). Both T-dependent (CD40) and T-independent (dual TLR-BCR, TACI-BCR or TLR-TACI engagement) CSR-inducing stimuli activate induction of AID and lead to class-switched B cells.

Ex vivo analysis of NSGW41 H-Mice B cells. Peripheral blood B cells from humanized mice were collected from H-Mice at 18 weeks of age. Surface expression of IgM, IgD, IgG, and IgA, as well CD19, CD27 and CD38 were analyzed by flow cytometry. CSR, CD27+ CD38+ plasma cells and CD27+ CD38− cells phenotypically resembling memory B cells were assessed.

The data also show the results of in vitro stimulation with CD154. Peripheral blood B cells from a healthy adult and humanized mice were stimulated for 96 h with CD154 plus IL-4 and IL-21. Surface expression of IgM, IgG, and IgA, as well CD19, CD27 and CD38 were analyzed by flow cytometry. CSR, CD27+ CD38+ plasma cells and CD27+ CD38− cells phenotypically resembling memory B cells were assessed.

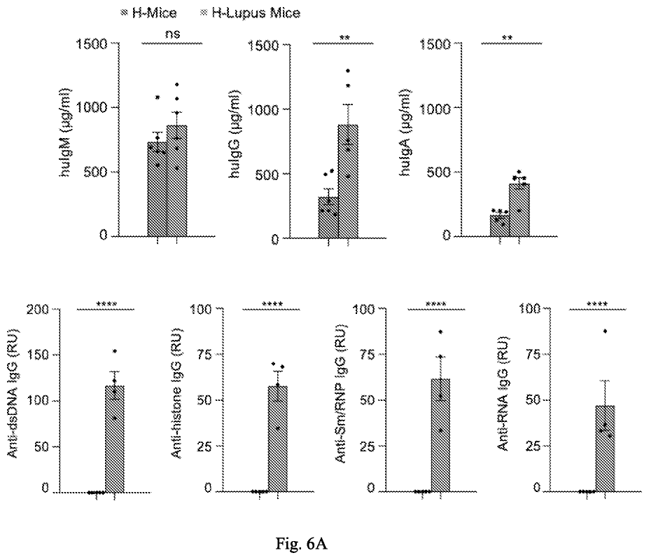

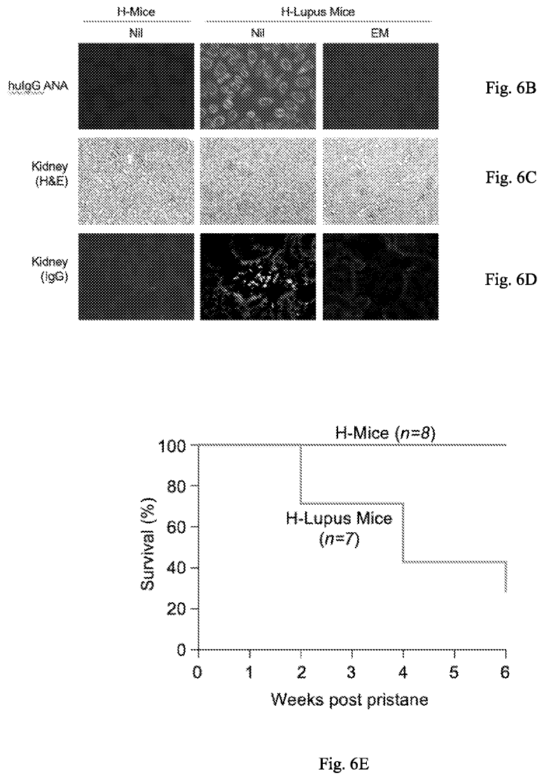

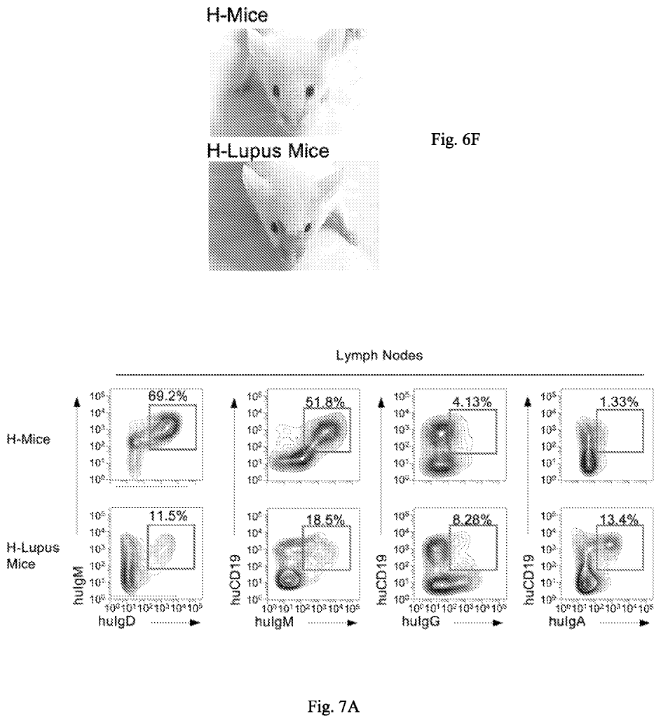

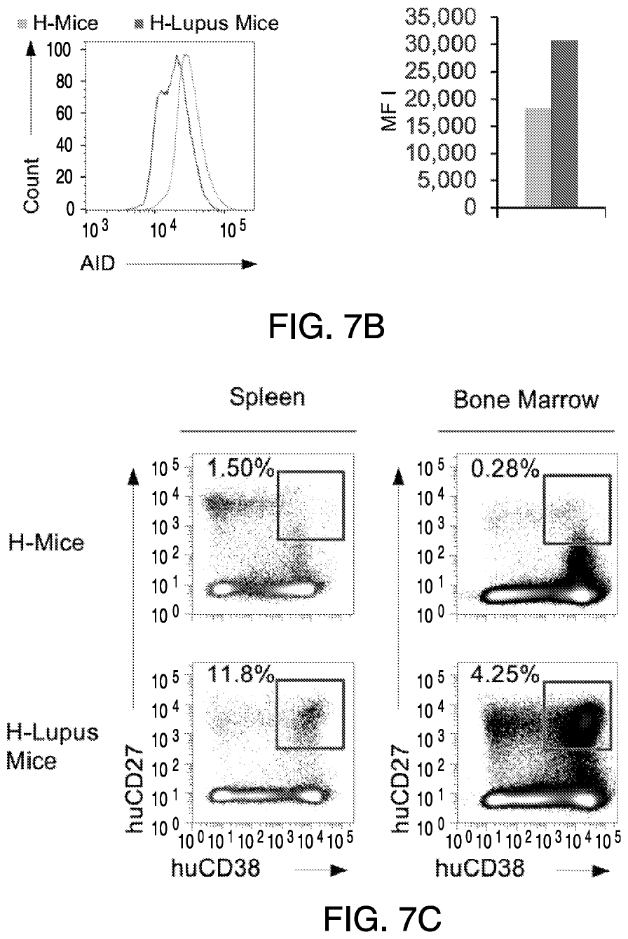

Pristane-induced H-Mice lupus model. Next, the NSGW41 H-Mice platform was applied to the study of autoimmune disease within the context of an in vivo human immune system. The ability to induce autoimmunity by i.p. injection of pristane into NSGW41 H-Mice at 20 weeks of age (H-Lupus Mice®) was investigated. Within 6 weeks post-injection, H-Lupus Mice® exhibited increased class-switched antibody titers and substantial IgG autoantibody production against dsDNA, histones, Sm/RNP, and RNA (FIG. 6A). This induction of autoimmunity led to IgG antinuclear antibodies and glomerulonephritis, characterized by glomerular enlargement, cellular deposition leading to severe disruption of the glomerular structur and IgG deposition (FIGS. 6B, C, D). H-Lupus Mice® have 70% mortality 6 weeks post-pristane (FIG. 6E) and develop severe facial dermatitis, recapitulating cutaneous lupus erythematosus (FIG. 6F). H-Lupus Mice® show increased peripheral blood class-switched B cells (FIG. 7A) concomitant with increased AID expression (FIG. 7A). These findings are paired with greater plasma cell differentiation in both the spleen and bone marrow of H-Lupus Mice® (FIG. 7C).

Conclusions. The results show that intracardiac transplant of CD34+ HSCs into immunodeficient NSGW41 mice leads to human engraftment levels reaching 95%. NSGW41 H-Mice have robust lymphatic development and differentiation of mature lymphocytes in peripheral blood and lymphatic tissues. B cells developed within NSGW41 H-Mice fully mature and respond to both T-dependent and T-independent antigens in vitro with CSR and plasma cell differentiation levels comparable to B cells from human adults. NSGW41 H-Mice respond to immunization with robust CSR, memory B cell generation, plasma cell differentiation and antigen specific antibody responses. Induction of autoimmunity in H-Mice generates symptoms modeling lupus. These findings demonstrate that a functional human immune system can be generated in vivo platform for translational humoral immunity research and vaccine development.

### Example 3

### Construction of Humanized Mice, Kinetics of Human Immune System Development and Induction of Matured Class-Switched Specific Antibody and Autoantibody Responses

Abstract. Inbred mice, the most widely used surrogates for human biology, recapitulate many features of the human immune system and immune response. They, however, diverge from humans in important immune elements and functions, such as differential TLR expression, certain antibody and T cell responses, species-specific viral and/or bacterial infections and drug interactions, thereby underscoring the need for a human model that mimics the in vivo human immune response. Humanized mice have recently provided some data on human infections, autoimmunity and cancer, with limited results. Described herein is a systematic approach to the generation of humanized mice by grafting NOD.Cg-Kit^{W-41J} Prkdc^{scid} Il2rg^{tm1Wjl}/WaskJ (NSG/Kit^{W41J}) mice within 48 hours of birth, devoid of prior irradiation, with human hematopoietic (CD34+) stem cells. Human umbilical cord hematopoietic stem cells were collected within 30 minutes post-partum and injected intracardially, consistently yielding up to 95% human cell peripheral reconstitution. Human leukocyte development, differentiation and long-term persistence in these CD34+ cell-grafted NSG/Kit^{W41J} (huNSG/Kit^{W41J}) mice revealed B and T cell maturation as part of a full immune system development, which led to emergence of IgM, IgG, IgA and IgE antibody titers comparable to wild type mice. The huNSG/Kit^{W41J} mice supported specific T-dependent and T-independent antibody responses that include human B cell class switch DNA recombination, somatic hypermutation, plasma cell and memory B cell differentiation. These mice also supported a hydrocarbon-induced autoantibody response leading to symptoms mimicking systemic lupus. Thus, these finding demonstrate the development of a robust in vivo platform allowing for generation and maturation of human antibody and autoantibody responses.

Collection of human umbilical cord blood samples. Cord blood samples are collected immediately following healthy, full-term cesarean section births. Samples are received within 20 minutes post-partum and hematopoietic stem cells are immediately isolated.

Isolation of hematopoietic stem cells. Hematopoietic stem cells are isolated from human umbilical cord blood by utilizing the StemCell EasySep CD34+ cord blood kit. Cord blood is pre-enriched for HSCs by RosetteSep antibody cocktail which depletes differentiated lymphocytes. Mononuclear cells are isolated from pre-enriched cord blood by density gradient centrifugation followed by positive selection of CD34+ cells. Isolation yields 1.25×106 HSC with 98% HSC purity or greater.

Engraftment of immunodeficient NSG and NSGW41 mice. Immunodeficient NSG and NSG/Kit^{W41J} are engrafted at 48 hours of age via intracardiac injection of 1.0×105 hematopoietic stem cells isolated from human umbilical cord blood.

NSGW41 H-Mice support greater engraftment and long-term persistence of human immune reconstitution than NSG mice. The data show that a human immune system can be developed within huNSG/KitW41J mice. Following intracardiac transplant of hematopoietic stem cells, human engraftment was assessed biweekly by flow cytometry analysis of PBMCs expressing either human or mouse CD45. Engraftment levels depicted as human CD45+ percent of total CD45+ cells. See also, FIG. 1F that shows the proportion of CD34+ cells within the bone marrow.

Human lymphocyte differentiation kinetics. Human leukocyte differentiation was assessed by flow cytometry analysis of PBMC surface expression of human CD45, CD19, CD3, CD11c, CD14 and CD56 in huNSG/KitW41J mice (see, for example, FIG. 1D, 1E). Spleen and lymph nodes were extracted at 25 weeks of age and percentages of CD3 and CD19 were assessed as percentage of total human CD45+ cells.

Ex vivo analysis of NSGW41 H-Mice B cells. Peripheral blood B cells from humanized mice were collected from H-Mice at 18 weeks of age. Surface expression of IgM, IgD, IgG, and IgA, as well CD19, CD27 and CD38 were analyzed by flow cytometry. CSR, CD27+ CD38+ plasma cells and CD27+ CD38− cells phenotypically resembling memory B cells were assessed.

B cells from huNSG/KitW41J mice undergo CSR in response to CD154 as efficiently as B cells from healthy human adults. Peripheral blood B cells from a healthy adult and huNSG/KitW41J mice were stimulated for 96 h with CD154 plus IL-4 and IL-21. Surface expression of IgM, IgG, and IgA, as well CD19, CD27 and CD38 were analyzed by flow cytometry. Proportions of CSR, and CD27+ CD38+ plasma cells were assessed.

B cells from huNSG/KitW41J mice undergo CSR and plasma cell differentiation in response to stimulation with TLR ligand. Peripheral blood B cells from a healthy adult and humanized mice were stimulated for 96 h with CpG plus IL-4 and IL-21. Surface expression of IgM, IgG, and IgA, as well CD19, CD27 and CD38 were analyzed by flow cytometry. CSR, CD27+ CD38+ plasma cells were assessed.

HuNSG/Kit^{W41J} mice injected with pristane develop a lupus-like disease. Briefly, huNSG/Kit^{W41J} mice were engrafted with human hematopoietic stem cells two days after birth. At 16 weeks of age huNSG/Kit^{W41J} mice were injected with 500 μl pristane (2,6,10,14-tetramethylpentadecane) i.p. Analysis of autoimmune symptoms was conducted 6 weeks post-pristane injection (week 19 after birth). FIG. 5 shows the titers of total human IgM, IgG and IgA as well as human anti-dsDNA IgG, anti-histone and anti-RNP autoantibodies analyzed by ELISA; autoantibody production assessed by ANA staining of serum collected from huNSG/Kit^{W41J} mice with and without pristane injection; and kidneys extracted from huNSG/KitW41J mice with and without pristane injection and stained with H&E.

Increased survival of lupus huNSG/Kit^{W41J} mice when treated with epigenetic regulator. FIG. 6 shows a reduced mortality and disease phenotype in huNSG/Kit^{W41J} mice receiving epigenetic modulator treatment. Beginning one week post-pristane injection, huNSG/Kit^{W41J} mice were given either normal drinking water or water with 0.3 mg/ml epigenetic regulator ad libitum and monitored for survival over 6 weeks following induction of autoimmunity.

Epigenetic regulation reduces AID expression, CSR and plasma differentiation in lupus huNSG/KitW41J mice. FIGS. 7A-D shows reduced CSR, AID expression and plasma cell differentiation in epigenetic regulator treated lupus huNSG/KitW41J mice. Beginning one week post-pristane injection huNSG/Kit^{W41J} mice were given either normal drinking water or water with 0.3 mg/ml epigenetic regulator ad libitum. Six weeks post-induction of autoimmunity mice were sacrificed.

Epigenetic regulation reduces autoantibody production and lupus nephritis in lupus huNSG/KitW41J mice. Epigenetic regulation reduces autoantibody production and lupus nephritis. Lupus huNSG/Kit^{W41J} mice were sacrificed 6 weeks post-induction of autoimmunity. Serum was collected and analyzed for antinuclear antibodies shown ANA staining. Kidney pathology was assessed by H&E and anti-human IgG staining of glomeruli.

Conclusions. These results show that intracardiac transplant of CD34+ HSCs into immunodeficient NSG/Kit^{W41J} mice leads to human engraftment levels reaching 95%. These findings demonstrate that HuNSG/Kit^{W41J} mice have robust lymphatic development and differentiation of mature lymphocytes in peripheral blood and lymphatic tissues. B cells developed within huNSG/Kit^{W41J} mice fully mature and respond to both T-dependent and T-independent antigens in vitro with CSR and plasma cell differentiation levels comparable to B cells from human adults. huNSG/Kit^{W41H} mice respond to immunization with robust CSR, memory B cell generation, plasma cell differentiation and antigen specific antibody responses. Induction of autoimmunity in huNSG/Kit^{W41J} mice generates symptoms modeling lupus. Autoantibody production and autoimmune pathology can be abrogated by epigenetic regulation.

What is claimed is:

1. A transgenic mouse, comprising: one or more human CD45 expressing cells, human B cells, human T cells, human dendritic cells, human monocytes/macrophages, human NK cells, human innate lymphoid cells, human microglia or human iNKT cells; and wherein the mouse's endogenous immune system is immunodeficient.

2. The transgenic mouse of claim 1, wherein the transgenic mouse comprises one or more mutations, wherein the one or more mutations is:
a) a loss of function mutation causing the mode of action (moa) loss-of-function mutation in the gene that encodes for the protein kinase, DNA-activated, catalytic polypeptide;
b) a loss-of-function mutation in the gene that encodes for the interleukin 2 receptor α; or
c) a loss-of-function mutation in a gene that encodes for a KIT receptor.

3. The transgenic mouse of any of claims 1-2, wherein the mouse further comprises an engraftment of human hematopoietic stem cells.

4. A transgenic mouse, comprising:
a) a loss-of-function mutation in the gene that encodes for the protein kinase, DNA-activated, catalytic polypeptide; and
b) a loss-of-function mutation in the gene that encodes for the interleukin 2 receptor α;
wherein the mouse further comprises an engraftment of human hematopoietic stem cells, and
wherein the mouse comprises one or more human CD45 expressing cells, human B cells, human T cells, human dendritic cells, human monocytes/macrophages, human NK cells, human innate lymphoid cells, human microglia and human iNKT cells.

5. The transgenic mouse of claim 4, wherein the mouse does not express a functional DNA-activated, catalytic polypeptide.

6. The transgenic mouse of claim any of claims 1-5, wherein the engraftment of human hematopoietic stem cells is through intracardial injection.

7. The transgenic mouse of claim 6, wherein the mouse does not express a functional interleukin 2 receptor α.

8. The transgenic mouse of claim 7, wherein the mouse comprises NOD.Cg-Kit^{W-41J} Prkdc^{scid} Il2rg^{tm1Wjl}WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

9. The transgenic mouse of any of claims 1-8, wherein the human hematopoietic stem cells comprise one or more cells selected from the group consisting of a human CD34-positive cell, a human hematopoietic stem cell, a human myeloid precursor cell, a human erythroid precursor cell, a human myeloid cell, a human dendritic cell, a human monocyte, a human granulocyte, a human erythrocyte, a human neutrophil, a human mast cell, a human thymocyte, and a human B lymphocyte.

10. The transgenic mouse of any of claims 1-9, wherein the hematopoietic stem cells are CD34+ stem cells.

11. The transgenic mouse of any of claims 1-10, wherein the mouse is treated with estrogen or estradiol.

12. The transgenic mouse of any of claims 1-11, wherein the mouse comprises two or more human CD45 expressing cells, human B cells, human T cells, human dendritic cells, human monocytes/macrophages, human NK cells, human innate lymphoid cells, human microglia and human iNKT cells.

13. The transgenic of claim 12, further comprising a loss-of-function mutation in a gene that encodes for a KIT receptor.

14. The transgenic mouse of claim 13, wherein the mouse does not express a functional KIT receptor.

15. The transgenic mouse of any of claims 1-14, wherein the mouse has a functional human immune system.

16. The transgenic mouse of any of claims 1-15, wherein the loss-of-function mutation in the gene that encodes for the protein kinase, DNA-activated, catalytic polypeptide comprises a T to A transversion point mutation at a position corresponding to codon 4046.

17. The transgenic mouse of any of claims 1-16, wherein the loss-of-function mutation in the gene that encodes for the protein kinase, DNA-activated, catalytic polypeptide is Prkdc^{scid}.

18. The transgenic mouse of any of claims 1-17, wherein the loss-of-function mutation in the gene that encodes for the interleukin 2 receptor α comprises a neomycin resistance cassette.

19. The transgenic mouse of any of claims 1-18, wherein the loss-of-function mutation in the gene that encodes for the interleukin 2 receptor α is Il2rg^{tm1Wjl}.

20. The transgenic mouse of any of claims 13-19 wherein the loss-of-function mutation in the gene that encodes for the KIT receptor comprises a G to A point mutation in a kinase domain at nucleotide 2519.

21. The transgenic mouse of any of claims 13-20, wherein the loss-of-function mutation in the gene that encodes for the KIT receptor is Cg-Kit^{W-41J}.

22. The transgenic mouse of any of claims 1-21, wherein the mouse comprises NOD.Cg-Kit^{W-41J} Prkdc^{scid} Il2rg^{tm1Wjl}/WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

23. The transgenic mouse of any of claims 1-22, wherein the mouse comprises mature human leukocytes.

24. The transgenic mouse of any of claims 1-23, wherein the mouse comprises one or more human hematopoietic lineage cells.

25. The transgenic mouse of claim 24, wherein the one or more human hematopoietic lineage cells is a B cell, a T cell, a monocyte, a macrophage, a dendritic cell, a NK cell, a iNKT cell, an innate lymphoid cell, a microglia or a red blood cell.

26. The transgenic mouse of claim 25, wherein the mouse comprises all human hematopoietic lineage cells.

27. The transgenic mouse of any of claims 25-26, wherein the one or more human hematopoietic lineage cells are maintained up to 40 weeks.

28. The transgenic mouse of any of claims 1-27, wherein the mouse comprises 95% human cell peripheral reconstitution.

29. The transgenic mouse of any of claims 1-28, wherein the mouse is capable of supporting full human leukocyte development, differentiation and persistence beyond 1 year of age without developing xeno-reactive graft-versus-host reaction and disease.

30. The transgenic mouse of any of claims 1-27, wherein the engrafted hematopoietic stem cells are capable of developing into one or more of a human B cell, a human T cell, a human monocyte, a human macrophage, a human dendritic cell, a human NK cell, a human iNKT cell, a human innate lymphoid cell, a human microglia and a human red blood cell or a combination thereof.

31. The transgenic mouse of of claim 30, wherein the mouse comprises at least one of each of a human B cell, a human T cell, a human monocyte, a human macrophage, a human dendritic cell, a human NK cell, a human iNKT cell, a human innate lymphoid cell, a human microglia and a human red blood cell.

32. The transgenic mouse of claim 31, wherein the at least one of each of a human B cell, a human T cell, a human monocyte, a human macrophage, a human dendritic cell, a human NK cell, a human iNKT cell, a human innate lymphoid cell, a human microglia and a human red blood cell reaches 95% human reconstitution within bone marrow or 98% within a secondary lymphoid organ in response to estrogen stimulation.

33. The transgenic mouse of claim 32, wherein the secondary lymphoid organ is a spleen, a mesenteric lymph node or a gut-associated lymphoid tissue.

34. The transgenic mouse of any of claims 1-33, wherein the mouse was not irradiated.

35. The transgenic mouse of any of claims 1-34, wherein the mouse is capable of producing one or more antibodies.

36. The transgenic mouse of any of claims 1-35, wherein the mouse is capable of physiological development and rearrangement of human B cell and T cell receptors thereby generating a repertoire diversity comparable to a healthy adult human for producing one or more human antibodies against a broad range of antigens.

37. The transgenic mouse of any of claims 1-36, wherein the mouse is capable of producing human IgM, IgD, IgG, IgA or IgE antibody titers.

38. The transgenic mouse of claim 37, wherein the human IgM, IgD, IgG, IgA or IgE antibody titers are comparable to those in an adult human in response to estrogen stimulation.

39. The transgenic mouse of any of claims 1-38, wherein the mouse comprises or is capable of producing an expanded myeloid lineage and T lymphocyte compartments in response to estrogen stimulation.

40. The transgenic mouse of any of claims 1-39, wherein the mouse comprises or is capable of producing one or more human T memory cells.

41. The transgenic mouse of any of claims 1-40, wherein the mouse comprises or is capable of producing human immune system reconstitution of one or more mucosal sites.

42. The transgenic mouse of claim 41, wherein the one or more mucosal sites is in the lungs.

43. The transgenic mouse of any of claims 1-42, wherein the mouse comprises or is capable of undergoing an increased AID and BLIMP1 expression, antibody class-switch DNA recombination, affinity maturation, somatic hypermutation, and/or B memory cell generation and plasma cell differentiation in response to estrogen stimulation.

44. The transgenic mouse of any of claims 1-43, wherein the mouse is capable of supporting human B cell development and differentiation to the extent that B cells express AID and BLIMP1, undergo antibody class-switch DNA recombination and plasma cell differentiation in response to in vitro stimulation as efficiently as B cells isolated from a healthy adult donor.

45. The transgenic mouse of any of claims 1-44, wherein the mouse provides a renewable source of one or more human hematopoietic lineage cells, wherein the one or more human hematopoietic lineage cells are a human B cell, a human T cell, a human monocyte, a human macrophage, a human dendritic cell, a human NK cell, a human iNKT cell, a human innate lymphoid cell, a human microglia and a human red blood cell or a combination thereof.

46. The transgenic mouse of any of claims 1-45, wherein the mouse is capable of supporting in vivo induction and maturation of a T lymphocyte-dependent or a T lymphocyte-independent antibody response.

47. The transgenic mouse of claim 46, wherein the T lymphocyte-dependent or a T lymphocyte-independent antibody response is potentiated in response to estrogen.

48. The transgenic mouse of claim 47, wherein the antibody response has undergone one or more of an antibody class-switch DNA recombination, a somatic hypermutation, a plasma cell differentiation, a memory B cell differentiation, development of peripheral germinal center or germinal center-like structures or secondary lymphoid organizations.

49. The transgenic mouse of any of claims 1-48, wherein the mouse is capable of supporting a systemic autoantibody response, wherein the systemic autoantibody response is induced by pristane thereby resulting in systemic or organ-specific autoimmunity.

50. The transgenic mouse of claim 49, wherein the systemic or organ-specific autoimmunity is a systemic lupus erythematosus-like disease.

51. The transgenic mouse of claim 50, wherein the systemic lupus erythematosus-like disease comprises IgM, IgG, IgA and IgE autoantibodies, wherein the one or more autoantibodies are present in a kidney or glomerulonephritis.

52. The transgenic mouse of any of claims 1-51, wherein the mouse is capable of supporting induction of IgE-mediated hypersensitivity, wherein the IgE-mediated hypersensitivity yields an allergic response to a respiratory or an alimentary allergen.

53. The transgenic mouse of claim 52, wherein the respiratory or the alimentary allergen is a house-dust mite or a peanut.

54. The transgenic mouse of claim 52, wherein the IgE-mediated hypersensitivity is facilitated by estrogen administration.

55. The transgenic mouse of any of claims 1-54, wherein the mouse is capable of being induced to develop or support engraftment and rejection of a liquid or a solid tumor including patient-derived xenograft.

56. The transgenic mouse of any of claims 1-55, wherein the mouse is capable of supporting vaccine and therapeutic development through testing the efficacy of an immunogen to identify and target a defined lymphocyte subset, wherein the defined lymphocyte subset expresses an antigen receptor capable of inducing a protective humoral immune response.

57. The transgenic mouse of any of claims 1-56, wherein the mouse is capable of supporting development of one or more therapeutics for one or more autoimmune diseases or allergic diseases through testing the efficacy of a small molecule compound to identify and target defined lymphocyte surface, intracellular molecules or different cell subsets.

58. The transgenic mouse of any of claims 1-57, wherein the mouse is capable of generating a fully human monoclonal antibody.

59. The transgenic mouse of any of claims 1-58, wherein the mouse is capable of generating a hybridoma of a predetermined antibody isotype and specificity.

60. The transgenic mouse of any of claims 1-59, wherein the mouse is capable of supporting one or more human microbial infections.

61. The transgenic mouse of any of claims 1-60, further comprising one or more human non-hematopoietic stem cell progenitors.

62. The transgenic mouse of claim 61, wherein the one or more human non-hematopoietic stem cell progenitors is obtained from human cord Wharton's jelly or perivascular tissue.

63. The transgenic mouse of claim 61, wherein the one or more human non-hematopoietic stem cell progenitors are lymphoid tissue organizer cells, lymphoid tissue inducer cells, marginal reticular cells, follicular dendritic cells or fibroblastic reticular cell precursors.

64. A method of making a transgenic mouse with a human immune system, the method comprising: engrafting a mouse with human hematopoietic cells, wherein the engrafting is intracardial, wherein the mouse's endogenous immune system is immunodeficient and wherein the transgenic mouse comprises one or more human CD45 expressing cells, human B cells, human T cells, human dendritic cells, human monocytes/macrophages, human NK cells, human innate lymphoid cells, human microglia or human iNKT cells.

65. The method of claim 64, wherein the engrafted mouse comprises one or more mutations, wherein the one or more mutations is:
a) a loss of function mutation causing the moa loss-of-function mutation in the gene that encodes for the protein kinase, DNA-activated, catalytic polypeptide;
b) a loss-of-function mutation in the gene that encodes for the interleukin 2 receptor α; or
c) a loss-of-function mutation in a gene that encodes for a KIT receptor.

66. A method of making a transgenic mouse with a human immune system, the method comprising: engrafting a mouse with human hematopoietic cells, wherein the engrafting is intracardial, wherein the mouse comprises:
a) a loss-of-function mutation in the gene that encodes for the protein kinase, DNA-activated, catalytic polypeptide, and
b) a loss of function mutation in the gene that encodes for the interleukin 2 receptor α.

67. The method of any of claims 64-66, wherein the human hematopoietic cells have a purity of at least 95%.

68. The method of any of claims 64-67, further comprising engrafting one or more human non-hematopoietic stem cell progenitors.

69. The method of claim 68, wherein the one or more human non-hematopoietic stem cell progenitors are lymphoid tissue organizer cells, marginal reticular cells, follicular dendritic cells, fibroblastic reticular cell precursors or a combination thereof.

70. The method of any of claims 64-66, wherein the transgenic mouse does not express a functional DNA-activated, catalytic polypeptide.

71. The method of any of claims 64-66, wherein the transgenic mouse does not express a functional interleukin 2 receptor α.

72. The method of any of claims 64-71, wherein the transgenic mouse carries the strain NOD.Cg-Kit^{W-41J} Prkdc^{scid} Il2rg^{tm1Wjl}/WaskJ (NSGW41), NOD.Cg-Kit^{W-41J} Tyr⁺ Prkdc^{scid} Il2rg^{tm1Wjl}/Thom0J (NBSGW) or NOD.Cg-Prkdc^{scid}Il2rg^{tm1Wjl}/SzJ (NSG).

73. The method of any of claims 64-72, wherein the human hematopoietic stem cells comprise one or more cells selected from the group consisting of a human CD34-positive cell, a human hematopoietic stem cell, a human myeloid precursor cell, a human erythroid precursor cell, a human myeloid cell, a human dendritic cell, a human monocyte, a human granulocyte, a human erythrocyte, a human neutrophil, a human mast cell, a human thymocyte, and a human B lymphocyte.

74. The method of any of claims 64-73, wherein the hematopoietic stem cells are CD34+ stem cells.

75. The method of any of claims 64-74, wherein the engrafted mouse is treated with estrogen or estradiol between day 7 and 21.

76. The method any of claims 64-75, wherein the transgenic mouse comprises one or more human CD45 expressing cells, human B cells, human T cells, human dendritic cells, human monocytes/macrophages and human NK cells, human innate lymphoid cells, human microglia and human iNKT cells.

77. The method of any of claims 64-76, wherein the transgenic mouse further comprises a loss-of-function mutation in a gene that encodes for a KIT receptor.

78. The method of claim 77, wherein the transgenic mouse does not express a functional KIT receptor.

79. The method of any of claims 64-78, wherein the transgenic mouse has a functional human immune system.

80. The method of any of claims 64-79, wherein the loss-of-function mutation in the gene that encodes for the protein kinase, DNA-activated, catalytic polypeptide comprises a T to A transversion point mutation at a position corresponding to codon 4046.

81. The method of any of claims 64-80, wherein the loss-of-function mutation in the gene that encodes for the protein kinase, DNA-activated, catalytic polypeptide is Prkdc^{scid}.

82. The method of any of claims 64-81, wherein the loss-of-function mutation in the gene that encodes for the interleukin 2 receptor α comprises a neomycin resistance cassette.

83. The method of any of claims 64-82, wherein the loss-of-function mutation in the gene that encodes for the interleukin 2 receptor α is Il2rg^{tm1Wjl}.

84. The method of any of claims 77-83, wherein the loss-of-function mutation in the gene that encodes for the KIT receptor comprises a G to A point mutation in a kinase domain at nucleotide 2519.

85. The method of any of claims 77-83, wherein the loss-of-function mutation in the gene that encodes for the KIT receptor is Cg-Kit^{W-41J}.

86. The method of any of claims 64-85, wherein the mouse comprises NOD.Cg-Kit^{W-41J} Prkdc^{scid} Il2rg^{tm1Wjl}/WaskJ (NSGW41) or NOD.Cg-Prkdc^{scid}Il2rg^{tm1Wjl}/SzJ (NSG).

87. The method of any of claims 64-86, wherein the transgenic mouse comprises mature human leukocytes.

88. The method of any of claims 64-87, wherein the transgenic mouse comprises one or more human hematopoietic lineage cells.

89. The method of claim 88, wherein the one or more human hematopoietic lineage cells is a B cell, a T cell, a monocyte, a macrophage, a dendritic cell, a NK cell, a iNKT cell, an innate lymphoid cell, microglia or a red blood cell.

90. The method of claim 89, wherein the transgenic mouse comprises all human hematopoietic lineage cells.

91. The method of claim 88, wherein the one or more human hematopoietic lineage cells are maintained at least 40 weeks.

92. A method of producing one or more human immune cells, the method comprising administering estrogen or estradiol to the transgenic mouse of any of claim 1-10, or 12-22 between day 7 and 21.

93. A method of producing one or more human antibodies, the method comprising introducing at least one candidate antigen into the transgenic mouse of any of claims 1-34; and recovering B cells and antibody-producing cells from the transgenic mouse.

94. The method of claim 93, further comprising rendering the B cells and antibody-producing cells into a single cell suspension; and generating an immortalized cell line from the single cell suspension.

95. The method of claim 94, wherein the immortalized cell line is a hybridoma cell line.

96. A method of assessing a human immune response in a mouse, the method comprising:
a) exposing the transgenic mouse of any of claims 1-34 to a candidate antigen;
b) taking a biological sample from the transgenic mouse exposed to the candidate antigen;
c) analyzing the biological sample using an assay to measure the immune response in the transgenic mouse to the candidate antigen.

97. The transgenic mouse of claim 64, wherein the engraftment of the human hematopoietic stem cells comprises an insertion or a deletion of one or more human genes prior to engraftment.
